(19) United States
(12) Patent Application Publication
(10) Pub. No.: US 2026/0354728 A1
(43) Pub. Date: Nov. 12, 2026

(54) ECG SIGNAL VISUALIZATION

(71) Applicant: Medtronic, Inc., Minneapolis, MN (US)

(72) Inventors: Niranjan Chakravarthy, Singapore (SG); Yong K. Cho, Excelsior, MN (US)

(21) Appl. No.: 19/125,072

(22) PCT Filed: Oct. 12, 2023
(86) PCT No.: PCT/IB2023/060290

§ 371 Date: Apr. 28, 2025

Related U.S. Application Data
(60) Provisional application No. 63/381,540, filed on Oct. 28, 2022.

(51) Int. Cl. A61B 5/327; A61B 5/00; A61B 5/352; A61B 5/361; A61B 5/363

(52) U.S. Cl. CPC A61B 5/327; A61B 5/352; A61B 5/361; A61B 5/363; A61B 5/7203; A61B 5/725; A61B 5/726

(57) ABSTRACT

This disclosure is directed to devices, systems, and techniques for changing a visualization of a sensed electrocardiogram (ECG) signal. An example device includes a memory configured to store the sensed ECG signal and processing circuitry communicatively coupled to the memory. The processing circuitry is configured to receive the sensed ECG signal. The processing circuitry is configured to process at least one of low frequency noise or high frequency noise in the sensed ECG signal to generate a processed ECG signal. The processing circuitry is configured to output the processed ECG signal for visualization by a clinician for diagnosis of a cardiac episode.

This application claims the benefit of U.S. Provisional Patent Application Ser. No. 63/381,540, filed 28 Oct. 2022, the entire content of which is incorporated herein by reference.

### TECHNICAL FIELD

The disclosure relates generally to devices and, more particularly, devices configured to monitor electrocardiograms (ECGs).

### BACKGROUND

Some types of devices may be used to monitor one or more physiological parameters of a patient. These devices may include implantable medical devices (IMDs), wearable devices, or other external devices. Such devices may include, or may be part of a system that includes, sensors that sense signals associated with such physiological parameters, such as an ECG signal of a patient. Such devices may also analyze the sensed ECG signal to detect potential cardiac episodes and classify the potential cardiac episodes according to types of suspected cardiac episodes based on the sensed ECG signal.

### SUMMARY

A medical device, such as an IMD, may be configured to sense an ECG of a patient, analyze the sensed ECG signal to detect potential cardiac episodes, and classify the potential cardiac episodes by type. In order to confirm or deny a device-detected cardiac episode, the IMD may transmit the sensed ECG to an external device for review by a clinician or other reviewer. However, ECGs sensed by an IMD may include artifacts, such as low frequency noise, high frequency noise, a variation of R-wave amplitudes, and/or lower P-wave amplitudes, which may make it difficult for the clinician to analyze the sensed ECG signal to verify the device-detected cardiac episode or refute the device-detected cardiac episode.

Unlike traditional cardiac episode detection algorithms, this disclosure is directed to techniques for improving the visualization of a sensed ECG signal by processing the sensed ECG signal to generate a processed ECG signal. The processed ECG signal may make it easier for a clinician to analyze a morphology of the sensed ECG signal to diagnose a device-detected cardiac episode. As such, the techniques of this disclosure may improve the diagnosis of device-detected cardiac episodes, for example, by clinicians, which may lead to better treatment of cardiac patients and better patient outcomes.

In some examples, a system includes: a memory configured to store the sensed ECG signal; and processing circuitry communicatively coupled to the memory, the processing circuitry being configured to: receive the sensed ECG signal; process at least one of low frequency noise or high frequency noise in the sensed ECG signal to generate a processed ECG signal in which the at least one of the low frequency noise or the high frequency noise is mitigated, wherein the ECG signal includes a plurality of waves, and the processing of the at least one of the low frequency noise or the high frequency noise preserves a morphology of the waves, and wherein as part of processing the at least one of low frequency noise or high frequency noise in the sensed ECG signal, the processing circuitry is configured to determine one or more wavelet transforms to the ECG signal; and output the processed ECG signal with the at least one of the low frequency noise or the high frequency noise mitigated and the morphology of the waves preserved for visualization by a clinician for diagnosis of a cardiac episode.

In some examples, a method includes: receiving the sensed ECG signal; processing at least one of low frequency noise or high frequency noise in the sensed ECG signal to generate a processed ECG signal in which the at least one of the low frequency noise or the high frequency noise is mitigated, wherein the ECG signal includes a plurality of waves, and the processing of the at least one of the low frequency noise or the high frequency noise preserves a morphology of the waves, and wherein as part of processing the at least one of low frequency noise or high frequency noise in the sensed ECG signal, the processing circuitry is configured to determine one or more wavelet transforms to the ECG signal; and outputting the processed ECG signal with the at least one of the low frequency noise or the high frequency noise mitigated and the morphology of the waves preserved for visualization by a clinician for diagnosis by the clinician of a cardiac episode.

In some examples, a non-transitory computer-readable medium includes instructions, which when executed, cause processing circuitry to: receive a sensed ECG signal; process at least one of low frequency noise or high frequency noise in the sensed ECG signal to generate a processed ECG signal in which the at least one of the low frequency noise or the high frequency noise is mitigated, wherein the ECG signal includes a plurality of waves, and the processing of the at least one of the low frequency noise or the high frequency noise preserves a morphology of the waves, and wherein as part of processing the at least one of low frequency noise or high frequency noise in the sensed ECG signal, the processing circuitry is configured to determine one or more wavelet transforms to the ECG signal; and output the processed ECG signal for visualization by a clinician for diagnosis of a cardiac episode.

This summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 illustrates the environment of an example medical device system in conjunction with a patient, in accordance with one or more techniques of this disclosure.

FIG. 2 is a conceptual drawing illustrating an example configuration of the IMD of the medical device system of FIG. 1, in accordance with one or more techniques described herein.

FIG. 3 is a functional block diagram illustrating an example configuration of the IMD of FIGS. 1 and 2, in accordance with one or more techniques described herein.

FIGS. 4A and 4B are block diagrams illustrating two additional example IMDs that may be substantially similar to the IMD of FIGS. 1-3, but which may include one or more additional features, in accordance with one or more techniques described herein.

FIG. 5 is a block diagram illustrating an example configuration of components of the external device of FIG. 1, in accordance with one or more techniques of this disclosure.

FIG. 6 is a block diagram illustrating an example system that includes an access point, a network, external computing devices, such as a server, and one or more other computing devices, which may be coupled to the IMD of FIGS. 1-4, an external device, and processing circuitry via a network, in accordance with one or more techniques described herein.

FIGS. 7A-7C is a conceptual diagram illustrating example sensed ECG signals.

FIGS. 8A-8C are conceptual diagrams of a first high pass filtered ECG, a second high pass filtered ECG, and a linear detrending ECG, respectively.

FIG. 9 is a flow diagram illustrating example low frequency artifact denoising techniques according to one or more aspects of this disclosure.

FIG. 10 is a conceptual diagram of an example ECG signal having the baseline drift removed while retaining the underlying P-QRS-T morphologies.

FIG. 11 is a flow diagram illustrating example high-frequency artifact denoising according to one or more aspects of this disclosure.

FIG. 12 is a conceptual diagram of an example ECG signal indicating device-detected beat markers and pre-processed beat markers to line up with a QRS maximum slew location according to one or more aspects of this disclosure.

FIGS. 13A-13C are conceptual diagrams illustrating example processed ECG signals and sensed ECG signals according to one or more aspects of this disclosure.

FIGS. 14A-14B are flow diagrams illustrating example R-wave amplitude normalization techniques according to one or more aspects of this disclosure.

FIGS. 15A-15C are conceptual diagrams illustrating example processed ECG signals and corresponding sensed ECG signals according to one or more aspects of this disclosure.

FIG. 16 is a flow diagram illustrating example P-wave amplification techniques according to one or more aspects of this disclosure.

FIGS. 17A-17C are conceptual diagrams illustrating example processed ECGs and corresponding sensed ECGs according to one or more aspects of this disclosure.

FIG. 18 is a flow diagram of example visualization techniques according to one or more aspects of this disclosure.

FIG. 19 is another flow diagram of example visualization techniques according to one or more aspects of this disclosure.

Like reference characters denote like elements throughout the description and figures.

### DETAILED DESCRIPTION

Certain medical devices, such as insertable cardiac monitors (ICMs) or other IMDs, may sense and monitor an ECG of a patient. Such IMDs may be configured to detect a potential cardiac episode via the ECG. In some examples, when such an IMD detects a potential cardiac episode, the IMD may attempt to classify the type of cardiac episode. The IMD may transmit sensed ECGs associated with a detected potential cardiac episodes to another device for eventual review by a clinician to diagnose the potential cardiac episode. However, ECGs sensed by an IMD, such as an IMD designed to ensure very high arrhythmia detection sensitivity, may include low frequency noise (which may include baseline drift), high frequency noise (which may include muscle noise), varying R-wave amplitudes, and low amplitude P-waves, which may make it difficult for a clinician to diagnose potential cardiac episodes from IMD sensed ECGs. Unlike conventional approaches used to detect arrythmia (e.g., those which may be used by the IMD to detect a potential cardiac episode), the techniques of this disclosure modify the ECG waveform to improve the visualization of the ECG waveform by a human reviewer enabling easier clinician diagnoses of any detected potential cardiac episodes.

For example, visualizing R-waves is desirable for a clinician to interpret true asystole, bradycardia, and/or tachycardia. Visualizing R-waves and the ECG baseline for presence/absence of P-waves and flutter waves is desirable to interpret true atrial fibrillation (AF)/atrial flutter (AFL) and AV Blocks. Changes in R-wave amplitude levels, high frequency noise, baseline drift artifacts and ECG morphology due to a location of an IMD sensing the ECG signal, e.g., using subcutaneous electrodes, may make it difficult for the clinician to view R-waves and P-waves, especially in ECGs rendered to static views (e.g., pdf reports). For example, high frequency noise in an AF episode may obscure the ECG baseline and may make it difficult to ensure absence of P-waves to adjudicate true AF. As another example, in ECGs with large R-wave amplitude changes or low-frequency/baseline drift artifact, all the R-waves may not be readily visible, making it difficult to adjudicate for the presence of true pause/bradycardia. This may be exacerbated when ECG signals are rendered in static views (e.g., pdf reports) for review by a clinician. ECGs sensed by an IMD may tend to have more artifacts and/or noise and less consistent magnitude peaks and valleys than ECGs sensed using externally located sensors attached to skin of a patient. Therefore, improving the visualization of ECGs sensed by IMDs may be particularly desirable. Such an IMD may transmit a sensed ECG, for example, via an external computing device, such as a patient programmer, a smartphone, or the like, to a clinician computing device. Processing circuitry of the IMD, the external computing device, the clinician computing device, a cloud computing system, or any combination thereof, may apply the techniques of this disclosure to the sensed ECG signal to improve visualization of the ECG signal for the clinician to diagnose a patient cardiac condition. In this manner, a clinician may, in effect, analyze an ECG of a patient to diagnose a patient cardiac condition even if the patient is at home and did not come into a clinic or other medical facility to have an ECG taken.

This disclosure describes techniques for processing a sensed ECG signal to improve the visualization of elements within the sensed ECG signal to improve clinician diagnoses. Such elements may be used, for example, by a clinician, to diagnose a device-detected cardiac episode. These techniques may include signal denoising and/or transformation techniques for an ECG signal, such as an ECG sensed and transmitted from an ICM or other IMD, prior to output for visualization, for a clear or clearer visualization by a clinician to enable or improve arrhythmia interpretation (e.g., diagnosis of a cardiac episode, such as a device-detected cardiac episode).

The techniques of this disclosure may facilitate the visualization of elements of a sensed ECG signal, such as R-waves, P-waves, and other elements of the sensed ECG signal in a manner which improves the ability of a clinician to analyze the elements of the sensed ECG signal to diagnose a cardiac episode. For example, processing circuitry may generate a processed ECG signal based on the sensed ECG signal by any of, or any combination of, removing low frequency noise, removing high frequency noise, normalizing R-wave amplitude(s), or amplifying P-wave amplitude(s). In some examples, how the sensed ECG signal is processed may be based, at least in part, on a type of cardiac episode that the IMD detected.

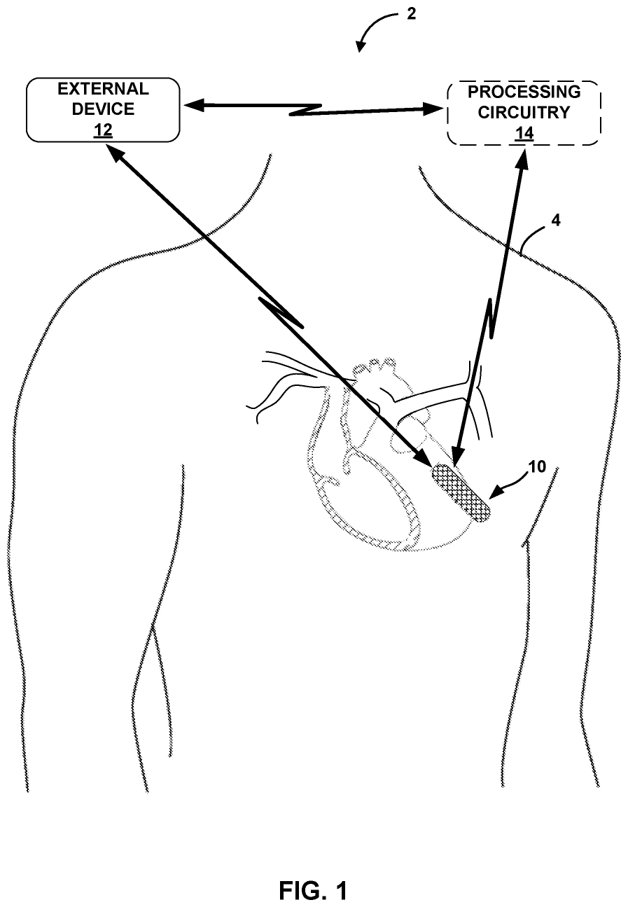

FIG. 1 illustrates the environment of an example medical device system 2 in conjunction with a patient 4, in accordance with one or more techniques of this disclosure. While the techniques described herein are generally described in the context of an ICM and/or an external device, the techniques of this disclosure may be implemented in any IMD or external device or combination thereof, capable of processing a sensed ECG signal. The example techniques may be used with an IMD 10, which may be in wireless communication with at least one of external device 12 and other devices not pictured in FIG. 1. Processing circuitry 14 is conceptually illustrated in FIG. 1 as separate from IMD 10 and external device 12, but may be processing circuitry of IMD 10 and/or processing circuitry of external device 12. In general, the techniques of this disclosure may be performed by processing circuitry 14 of one or more devices of a system, such as one or more devices that include sensors that provide signals, or processing circuitry of one or more devices that do not include sensors, but nevertheless process signals using the techniques described herein. For example, another external device (not pictured in FIG. 1) may include at least a portion of processing circuitry 14, the other external device configured for remote communication with IMD 10 and/or external device 12 via a network.

In some examples, IMD 10 is implanted outside of a thoracic cavity of patient 4 (e.g., subcutaneously in the pectoral location illustrated in FIG. 1). IMD 10 may be positioned near the sternum near or just below the level of patient 4's heart, e.g., at least partially within the cardiac silhouette. For other medical conditions, IMD 10 may be implanted in other appropriate locations, such as the interstitial space, abdomen, back of arm, wrist, etc. In some examples, IMD 10 takes the form of a LINQ™ Insertable Cardiac Monitor (ICM), available from Medtronic plc, of Dublin, Ireland.

Clinicians sometimes diagnose patients with medical conditions based on one or more observed physiological signals collected by physiological sensors, such as electrodes, optical sensors, chemical sensors, temperature sensors, acoustic sensors, and motion sensors. In some cases, clinicians apply non-invasive sensors to patients in order to sense one or more physiological signals while a patient is in a clinic for a medical appointment. However, in some examples, physiological markers (e.g., arrythmia, etc.) of a patient condition are rare or are difficult to observe over a relatively short period of time. As such, in these examples, a clinician may be unable to observe the physiological markers needed to diagnose a patient with a medical condition or effectively treat the patient while monitoring one or more physiological signals of the patient during a medical appointment.

In the example illustrated in FIG. 1, IMD 10 is implanted within patient 4 to continuously record one or more physiological signals, including an electrocardiogram (ECG). Other such physiological signals may include an electromyogram (EMG), impedance, respiration, activity, posture, blood oxygen saturation, or other physiological signals, of patient 4 over an extended period of time. In some examples, IMD 10 includes a plurality of electrodes. The plurality of electrodes is configured to detect signals that enable processing circuitry 14, e.g., of IMD 10, to monitor and/or record physiological parameters of patient 4. For example, the plurality of electrodes may be configured to sense an ECG of patient 4. IMD 10 may additionally or alternatively include one or more optical sensors, accelerometers, temperature sensors, chemical sensors, light sensors, pressure sensors, and/or respiratory sensors, in some examples. Such sensors may detect one or more physiological parameters indicative of a patient condition. In some examples, additional sensors may be located on other devices (not shown in FIG. 1) which may also sense physiological parameters of patient 4.

Sensor data may be collected by various devices such as implantable therapy devices, implantable monitoring devices, wearable devices, point of care devices, and noncontact sensors in the home or vehicle or other area frequented by the patient or a combination of such sensor platforms. The sensor data collected may be relevant to the disease state (e.g., heart failure) or comorbidities (e.g., chronic obstructive pulmonary disease (COPD), kidney disease, etc.) or for diagnosing a suspected comorbidity. For patients with multiple comorbidities, it may be possible to perform a differential diagnosis between different sources of a problem, e.g., heart failure decompensation, COPD exacerbation, pneumonia, etc. This would permit a clinician to prescribe appropriate therapies to address the patient's condition, such as diuretics, antibiotics, encourage fluids, etc.

Processing circuitry 14 may be configured to receive a sensed ECG signal, for example, from sensing circuitry of IMD 10. In some examples, processing circuitry 14 may analyze the sensed ECG signal to detect a cardiac episode of patient 4 and may classify the cardiac episode according to a type. Examples of such types include pause, AF, tachycardia, bradycardia, AT, etc. Processing circuitry 14 may be configured to generate a processed ECG signal based on the sensed ECG signal which may improve visualization for a clinician to assist in diagnosing a device-detected cardiac episode. For example, processing circuitry 14 may output, for visualization, a representation of the processed ECG signal, e.g., for static viewing (such as a pdf) or for dynamic viewing (such as a video). Such output may be to a user interface, a display device, a printed document, or any other visible form.

In some examples, the generation of the processed ECG signal may occur within IMD 10, within external device 12, within a cloud computing environment, or any combination thereof. For example, processing circuitry 14 generate the processed ECG signal by removing low frequency noise, removing high frequency noise, normalizing R-wave amplitude(s), and/or amplifying P-wave amplitude(s).

External device 12 may be a hand-held computing device with a display viewable by the user and an interface for providing input to external device 12 (e.g., a user input mechanism). For example, external device 12 may include a display screen (e.g., a liquid crystal display (LCD) or a light emitting diode (LED) display) that presents information to the user.

In addition, external device 12 may include a touch screen display, keypad, buttons, a peripheral pointing device, voice activation, or another input mechanism that allows the user to navigate through the user interface of external device 12 and provide input. If external device 12 includes buttons and a keypad, the buttons may be dedicated to performing a certain function, e.g., a power button, the buttons and the keypad may be soft keys that change in function depending upon the section of the user interface currently viewed by the user, or any combination thereof.

In some examples, external device 12 may be a separate application within another multi-function device, rather than a dedicated computing device. For example, the multi-function device may be a cellular phone, a tablet computer, a digital camera, or another computing device that may run an application that enables external device to operate as described herein.

When external device 12 is configured for use by the clinician, external device 12 may be used to transmit instructions to IMD 10 and to receive measurements, such sensed ECG signals, or the like. Example instructions may include requests to set electrode combinations for sensing and any other information that may be useful for programming into IMD 10. The clinician may also configure and store operational parameters for IMD 10 within IMD 10 with the aid of external device 12. In some examples, external device 12 assists the clinician in the configuration of IMD 10 by providing a system for identifying potentially beneficial operational parameter values.

Whether external device 12 is configured for clinician or patient use, external device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth® specification sets, or other communication technologies operable at ranges greater than near-field communication technologies).

Processing circuitry 14, in some examples, may include one or more processors that are configured to implement functionality and/or process instructions for execution within IMD 10 and/or external device 12. For example, processing circuitry 14 may be capable of processing instructions stored in a storage device. Processing circuitry 14 may include, for example, microprocessors, digital signal processors (DSPs), application specific integrated circuits (ASICs), field-programmable gate arrays (FPGAs), or equivalent discrete or integrated logic circuitry, or a combination of any of the foregoing devices or circuitry. Accordingly, processing circuitry 14 may include any suitable structure, whether in hardware, software, firmware, or any combination thereof, to perform the functions ascribed herein to processing circuitry 14.

Processing circuitry 14 may represent processing circuitry located within any combination of IMD 10 and/or external device 12. In some examples, processing circuitry 14 may be entirely located within a housing of IMD 10. In other examples, processing circuitry 14 may be entirely located within a housing of external device 12. In other examples, processing circuitry 14 may be located within any one of or a combination of IMD 10, external device 12, and another device or group of devices that are not illustrated in FIG. 1, e.g., a cloud computing environment. As such, techniques and capabilities attributed herein to processing circuitry 14 may be attributed to any combination of IMD 10, external device 12, and other devices that are not illustrated in FIG. 1.

Although in one example, IMD 10 takes the form of an ICM, in other examples, IMD 10 takes the form of any one or more of an ICM, a pacemaker, a defibrillator, a cardiac resynchronization therapy device, an implantable pulse generator, an intra-cardiac pressure measuring device, a ventricular assist device, a pulmonary artery pressure device, a subcutaneous blood pressure device, or the like. The physiological parameters may be sensed or determined using one or more of the aforementioned devices, as well as external devices such as external device 12.

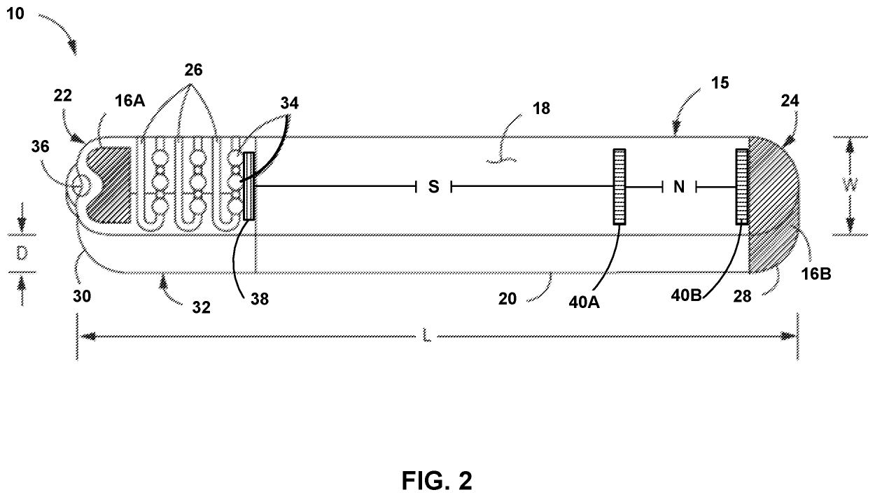

FIG. 2 is a conceptual drawing illustrating an example configuration of IMD 10 of the medical device system 2 of FIG. 1, in accordance with one or more techniques described herein. In the example shown in FIG. 2, IMD 10 may be a leadless, vascularly-implantable monitoring device having housing 15, proximal electrode 16A, and distal electrode 16B. Housing 15 may further include first major surface 18, second major surface 20, proximal end 22, and distal end 24. In some examples, IMD 10 may include one or more additional electrodes 16C, 16D positioned on one or both of major surfaces 18, 20 of IMD 10. Housing 15 encloses electronic circuitry located inside the IMD 10, and protects the circuitry contained therein from fluids such as body fluids (e.g., blood). In some examples, electrical feedthroughs provide electrical connection of electrodes 16A-16D, and antenna 26, to circuitry within housing 15. In some examples, electrode 16B may be formed from an uninsulated portion of conductive housing 15.

In the example shown in FIG. 2, IMD 10 is defined by a length L, a width W, and thickness or depth D. In this example, IMD 10 is in the form of an elongated rectangular prism in which length L is significantly greater than width W, and in which width W is greater than depth D. However, other configurations of IMD 10 are contemplated, such as those in which the relative proportions of length L, width W, and depth D vary from those described and shown in FIG. 2. In some examples, the geometry of the IMD 10, such as the width W being greater than the depth D, may be selected to allow IMD 10 to be inserted under the skin of the patient using a minimally invasive procedure and to remain in the desired orientation during insertion. In addition, IMD 10 may include radial asymmetries (e.g., the rectangular shape) along a longitudinal axis of IMD 10, which may help maintain the device in a desired orientation following implantation.

In some examples, a spacing between proximal electrode 16A and distal electrode 16B may range from about 30-55 mm, about 35-55 mm, or about 40-55 mm, or more generally from about 25-60 mm. Overall, IMD 10 may have a length L of about 20-30 mm, about 40-60 mm, or about 45-60 mm. In some examples, the width W of major surface 18 may range from about 3-10 mm, and may be any single width or range of widths between about 3-10 mm. In some examples, a depth D of IMD 10 may range from about 2-9 mm. In other examples, the depth D of IMD 10 may range from about 2-5 mm, and may be any single or range of depths from about 2-9 mm. In any such examples, IMD 10 is sufficiently compact to be implanted within the subcutaneous space of patient 4 in the region of a pectoral muscle.

IMD 10, according to an example of the present disclosure, may have a geometry and size designed for ease of implant and patient comfort. Examples of IMD 10 described in this disclosure may have a volume of 3 cubic centimeters (cm³) or less, 1.5 cm³ or less, or any volume therebetween. In addition, in the example shown in FIG. 2, proximal end 22 and distal end 24 are rounded to reduce discomfort and irritation to surrounding tissue once implanted under the skin of patient 4.

In the example shown in FIG. 2, first major surface 18 of IMD 10 faces outward towards the skin, when IMD 10 is inserted within patient 4, whereas second major surface 20 is faces inward toward musculature of patient 4. Thus, first and second major surfaces 18, 20 may face in directions along a sagittal axis of patient 4 (see FIG. 1), and this orientation may be generally maintained upon implantation due to the dimensions of IMD 10.

Proximal electrode 16A and distal electrode 16B may be used to sense cardiac EGM signals (e.g., ECG signals) when IMD 10 is implanted subcutaneously in patient 4. In some examples, processing circuitry of IMD 10 also may detect a suspected cardiac episode based on the sensed ECG signals of patient 4. In some examples, processing circuitry of IMD 10 may determine a type of the suspected cardiac episode. The cardiac ECG signals may be stored in a memory of IMD 10, and data derived from the cardiac ECG signals and/or other sensor signals, such as the type of suspected cardiac episode and the cardiac ECG signals may be transmitted via integrated antenna 26 to another device, such as external device 12. Additionally, in some examples, electrodes 16A, 16B may be used by communication circuitry of IMD 10 for tissue conductance communication (TCC) communication with external device 12 or another device. For example, external device 12 or another device may process the received ECG signals to improve the visualization of features of the sensed ECG signals and either display, or otherwise present (e.g., print out), the processed ECG signals for review by a clinician. As such, a clinician may be better able to accurately diagnose a cardiac episode.

In the example shown in FIG. 2, proximal electrode 16A is in close proximity to proximal end 22, and distal electrode 16B is in close proximity to distal end 24 of IMD 10. In this example, distal electrode 16B is not limited to a flattened, outward facing surface, but may extend from first major surface 18, around rounded edges 28 or end surface 30, and onto the second major surface 20 in a three-dimensional curved configuration. As illustrated, proximal electrode 16A is located on first major surface 18 and is substantially flat and outward facing. However, in other examples not shown here, proximal electrode 16A and distal electrode 16B both may be configured like proximal electrode 16A shown in FIG. 2, or both may be configured like distal electrode 16B shown in FIG. 2. In some examples, additional electrodes 16C and 16D may be positioned on one or both of first major surface 18 and second major surface 20, such that a total of four electrodes are included on IMD 10. Any of electrodes 16A-16D may be formed of a biocompatible conductive material. For example, any of electrodes 16A-16D may be formed from any of stainless steel, titanium, platinum, iridium, or alloys thereof. In addition, electrodes of IMD 10 may be coated with a material such as titanium nitride or fractal titanium nitride, although other suitable materials and coatings for such electrodes may be used.

In the example shown in FIG. 2, proximal end 22 of IMD 10 includes header assembly 32 having one or more of proximal electrode 16A, integrated antenna 26, anti-migration projections 34, and suture hole 36. Integrated antenna 26 is located on the same major surface (e.g., first major surface 18) as proximal electrode 16A, and may be an integral part of header assembly 32. In other examples, integrated antenna 26 may be formed on the major surface opposite from proximal electrode 16A, or, in still other examples, may be incorporated within housing 15 of IMD 10. Antenna 26 may be configured to transmit or receive electromagnetic signals for communication. For example, antenna 26 may be configured to transmit to or receive signals from a programmer (e.g., external device 12) via inductive coupling, electromagnetic coupling, tissue conductance, Near Field Communication (NFC), Radio Frequency Identification (RFID), Bluetooth®, WiFi®, or other proprietary or non-proprietary wireless telemetry communication schemes. Antenna 26 may be coupled to communication circuitry of IMD 10, which may drive antenna 26 to transmit signals to external device 12, and may transmit signals received from external device 12 to processing circuitry of IMD 10 via communication circuitry.

In some examples, IMD 10 may include several features for retaining IMD 10 in position once subcutaneously implanted in patient 4, so as to decrease the chance that IMD 10 migrates in the body of patient 4. For example, as shown in FIG. 2, housing 15 may include anti-migration projections 34 positioned adjacent integrated antenna 26. Anti-migration projections 34 may include a plurality of bumps or protrusions extending away from first major surface 18, and may help prevent longitudinal movement of IMD 10 after implantation in patient 4. In other examples, anti-migration projections 34 may be located on the opposite major surface as proximal electrode 16A and/or integrated antenna 26. In addition, in the example shown in FIG. 2 header assembly 32 includes suture hole 36, which provides another means of securing IMD 10 to the patient to prevent movement following insertion. In the example shown, suture hole 36 is located adjacent to proximal electrode 16A. In some examples, header assembly 32 may include a molded header assembly made from a polymeric or plastic material, which may be integrated or separable from the main portion of IMD 10.

In the example shown in FIG. 2, IMD 10 includes a light emitter 38, a proximal light detector 40A, and a distal light detector 40B positioned on housing 15 of IMD 10. Light detector 40A may be positioned at a distance S from light emitter 38, and a distal light detector 40B positioned at a distance S+N from light emitter 38. In other examples, IMD 10 may include only one of light detectors 40A, 40B, or may include additional light emitters and/or additional light detectors. Although light emitter 38 and light detectors 40A, 40B are described herein as being positioned on housing 15 of IMD 10, in other examples, one or more of light emitter 38 and light detectors 40A, 40B may be positioned, on a housing of another type of IMD within patient 4, such as a transvenous, subcutaneous, or extravascular pacemaker or ICD, or connected to such a device via a lead.

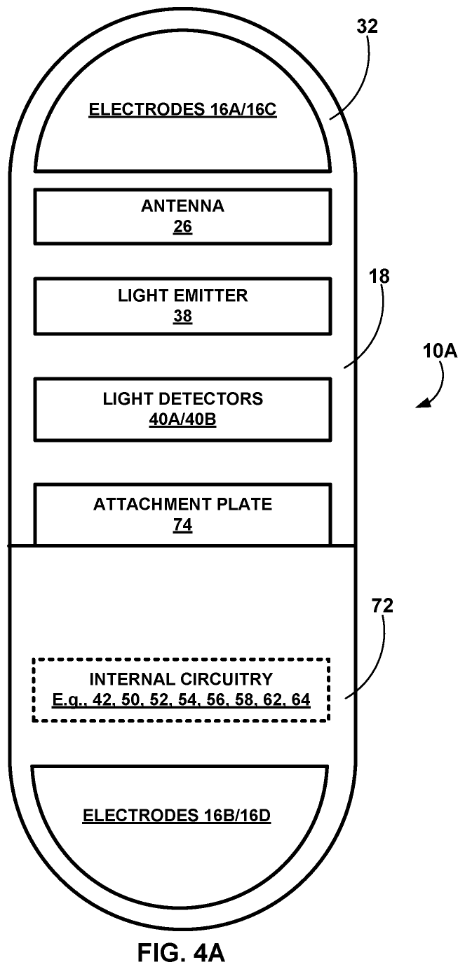

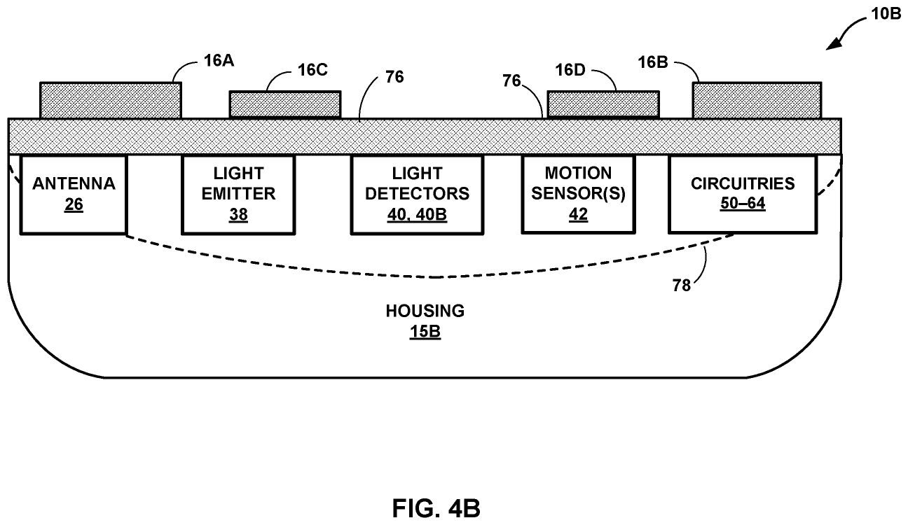

As shown in FIG. 2, light emitter 38 may be positioned on header assembly 32, although, in other examples, one or both of light detectors 40A, 40B may additionally or alternatively be positioned on header assembly 32. In some examples, light emitter 38 may be positioned on a medial section of IMD 10, such as part way between proximal end 22 and distal end 24. Although light emitter 38, and light detectors 40A, 40B, are illustrated as being positioned on first major surface 18, light emitter 38, and light detectors 40A, 40B, alternatively may be positioned on second major surface 20. In some examples, IMD may be implanted such that light emitter 38 and light detectors 40A, 40B face inward when IMD 10 is implanted, toward the muscle of patient 4, which may help minimize interference from background light coming from outside the body of patient 4. Light detectors 40A, 40B may include a glass or sapphire window, such as described below with respect to FIG. 4B, or may be positioned beneath a portion of housing 15 of IMD 10 that is made of glass or sapphire, or otherwise transparent or translucent.

In some examples, IMD 10 may include one or more additional sensors, such as one or more motion sensors (not shown in FIG. 2). Such motion sensors may be 3D accelerometers configured to generate signals indicative of one or more types of movement of the patient, such as gross body movement (e.g., motion) of the patient, patient posture, movements associated with the beating of the heart, or coughing, rales, or other respiration abnormalities, or the movement of IMD 10 within the body of patient 4. One or more of the parameters monitored by IMD 10 (e.g., bio impedance, respiration rate, EGM, etc.) may fluctuate in response to changes in one or more such types of movement. For example, changes in parameter values sometimes may be attributable to increased patient motion (e.g., exercise or other physical motion as compared to immobility) or to changes in patient posture, and not necessarily to changes in a medical condition. Thus, in some methods of identifying or tracking a medical condition of patient 4, it may be advantageous to account for such fluctuations when determining whether a change in a parameter is indicative of a change in a medical condition.

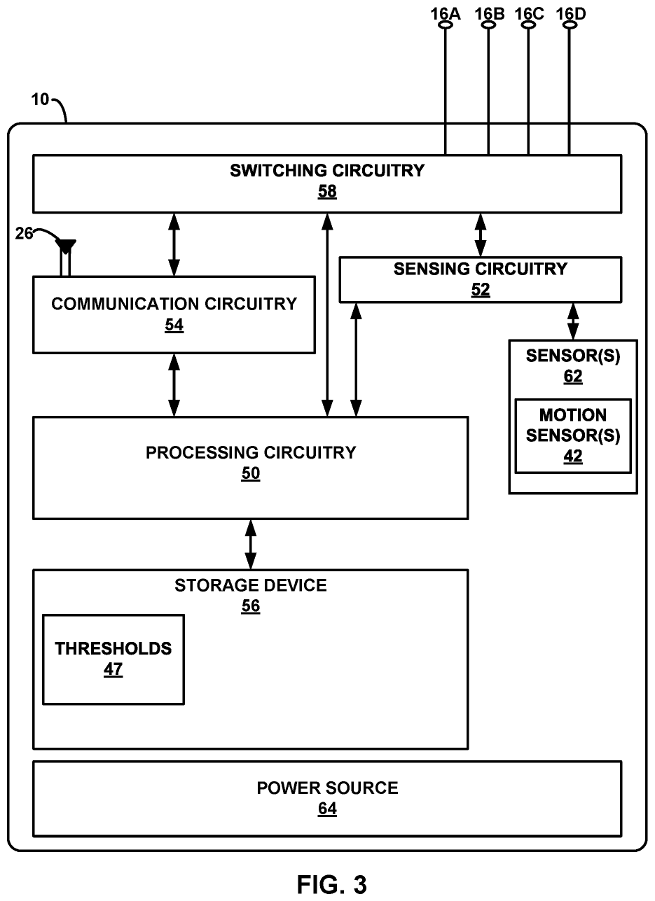

FIG. 3 is a functional block diagram illustrating an example configuration of IMD 10 of FIGS. 1 and 2, in accordance with one or more techniques described herein. In the illustrated example, IMD 10 includes electrodes 16, antenna 26, processing circuitry 50, sensing circuitry 52, communication circuitry 54, storage device 56, switching circuitry 58, sensors 62 including motion sensor(s) 42 (which may include an accelerometer), and power source 64.

Processing circuitry 50 may include fixed function circuitry and/or programmable processing circuitry. Processing circuitry 50 may include any one or more of a microprocessor, a controller, a DSP, an ASIC, an FPGA, or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof. In some examples, one or more techniques of this disclosure may be performed by processing circuitry 50.

Sensing circuitry 52 and communication circuitry 54 may be selectively coupled to electrodes 16A-16D via switching circuitry 58, as controlled by processing circuitry 50. Sensing circuitry 52 may monitor signals from electrodes 16A-16D in order to monitor electrical activity of heart (e.g., to produce a sensed ECG signal). Sensing circuitry 52 also may monitor signals from sensors 62, which may include motion sensor(s) 42 (which may include an accelerometer). In some examples, sensing circuitry 52 may include one or more filters and amplifiers for filtering and amplifying signals received from one or more of electrodes 16A-16D and/or motion sensor(s) 42.

Communication circuitry 54 may include any suitable hardware, firmware, software or any combination thereof for communicating with another device, such as external device 12 or another IMD or sensor, such as a pressure sensing device. Under the control of processing circuitry 50, communication circuitry 54 may receive downlink telemetry from, as well as send uplink telemetry to, external device 12 or another device with the aid of an internal or external antenna, e.g., antenna 26 (FIG. 2). In addition, processing circuitry 50 may communicate with a networked computing device via an external device (e.g., external device 12) and a computer network, such as the Medtronic CareLink® Network developed by Medtronic, plc, of Dublin, Ireland.

A clinician or other user may retrieve data from IMD 10 using external device 12, or by using another local or networked computing device configured to communicate with processing circuitry 50 via communication circuitry 54. The clinician may also program parameters of IMD 10 using external device 12 or another local or networked computing device.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), ferroelectric RAM (FRAM) read-only memory (ROM), non-volatile RAM (NVRAM), electrically-erasable programmable ROM (EEPROM), flash memory, or any other digital media.

Power source 64 is configured to deliver operating power to the components of IMD 10. Power source 64 may include a battery and a power generation circuit to produce the operating power. In some examples, the battery is rechargeable to allow extended operation. In some examples, recharging is accomplished through proximal inductive interaction between an external charger and an inductive charging coil within external device 12. Power source 64 may include any one or more of a plurality of different battery types, such as nickel cadmium batteries and lithium ion batteries. A non-rechargeable battery may be selected to last for several years, while a rechargeable battery may be inductively charged from an external device, e.g., on a daily or weekly basis.

FIGS. 4A and 4B illustrate two additional example IMDs that may be substantially similar to IMD 10 of FIGS. 1-3, but which may include one or more additional features, in accordance with one or more techniques described herein. The components of FIGS. 4A and 4B may not necessarily be drawn to scale, but instead may be enlarged to show detail. FIG. 4A is a block diagram of a top view of an example configuration of an IMD 10A. FIG. 4B is a block diagram of a side view of example IMD 10B, which may include an insulative layer as described below.

FIG. 4A is a conceptual drawing illustrating another example IMD 10A that may be substantially similar to IMD 10 of FIG. 1. In addition to the components illustrated in FIGS. 1-3, the example of IMD 10 illustrated in FIG. 4A also may include a body portion 72, an attachment plate 74, and treatment 45. Attachment plate 74 may be configured to mechanically couple header assembly 32 to body portion 72 of IMD 10A. Body portion 72 of IMD 10A may be configured to house one or more of the internal components of IMD 10 illustrated in FIG. 3, such as one or more of processing circuitry 50, sensing circuitry 52, communication circuitry 54, storage device 56, switching circuitry 58, internal components of sensors 62, and power source 64. In some examples, body portion 72 may be formed of one or more of titanium, ceramic, or any other suitable biocompatible materials.

FIG. 4B is a conceptual drawing illustrating another example IMD 10B that may include components substantially similar to IMD 10 of FIG. 1. In addition to the components illustrated in FIGS. 1-3, the example of IMD 10B illustrated in FIG. 4B also may include a wafer-scale insulative cover 76, which may help insulate electrical signals passing between electrodes 16A-16D, light detectors 40A, 40B on housing 15B and processing circuitry 50. In some examples, insulative cover 76 may be positioned over an open housing 15 to form the housing for the components of IMD 10B. One or more components of IMD 10B (e.g., antenna 26, light emitter 38, light detectors 40A, 40B, processing circuitry 50, sensing circuitry 52, communication circuitry 54, switching circuitry 58, and/or power source 64) may be formed on a bottom side of insulative cover 76, such as by using flip-chip technology. Insulative cover 76 may be flipped onto a housing 15B. When flipped and placed onto housing 15B, the components of IMD 10B formed on the bottom side of insulative cover 76 may be positioned in a gap 78 defined by housing 15B.

Insulative cover 76 may be configured so as not to interfere with the operation of IMD 10B. For example, one or more of electrodes 16A-16D may be formed or placed above or on top of insulative cover 76, and electrically connected to switching circuitry 58 through one or more vias (not shown) formed through insulative cover 76. Insulative cover 76 may be formed of sapphire (e.g., corundum), glass, parylene, and/or any other suitable insulating material. Sapphire may be greater than 80% transmissive for wavelengths in the range of about 300 nm to about 4000 nm, and may have a relatively flat profile. In the case of variation, different transmissions at different wavelengths may be compensated for, such as by using a ratiometric approach. In some examples, insulative cover 76 may have a thickness of about 300 micrometers to about 600 micrometers. Housing 15B may be formed from titanium or any other suitable material (e.g., a biocompatible material), and may have a thickness of about 200 micrometers to about 500 micrometers. These materials and dimensions are examples only, and other materials and other thicknesses are possible for devices of this disclosure.

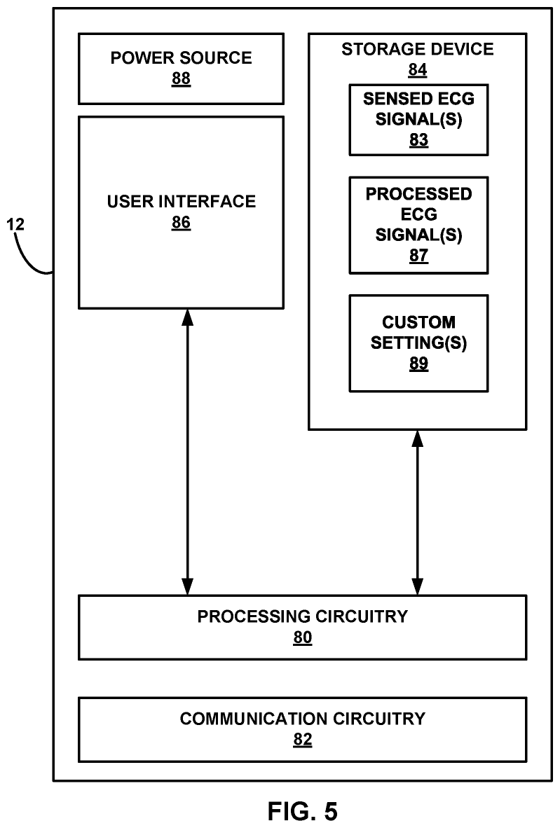

FIG. 5 is a block diagram illustrating an example configuration of components of external device 12, in accordance with one or more techniques of this disclosure. In the example of FIG. 5, external device 12 includes processing circuitry 80, communication circuitry 82, storage device 84, user interface 86, and power source 88. In some examples, external device 12 may include additional components not depicted in FIG. 5 or fewer components than depicted in FIG. 5.

Processing circuitry 80, in one example, may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80. In some examples, processing circuitry 80 may perform one or more of the techniques of this disclosure.

Processing circuitry 80 may receive a sensed ECG signal. In some examples, processing circuitry 80 may process at least one of low frequency noise or high frequency noise in the sensed ECG signal to generate a processed ECG signal. In some examples, processing circuitry 80 may, at least one of, normalize at least one R-wave of the sensed ECG signal or amplify at least one P-wave of the sensed ECG signal to generate a processed ECG signal. Processing circuitry 80 may output the processed ECG signal for visualization by a clinician for diagnosis of a cardiac episode.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. For example, communication circuitry 82 may receive a sensed ECG signal from IMD 10 and/or a type of a suspected cardiac episode.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, dynamic random access memories (DRAM), static random access memories (SRAM), magnetic discs, optical discs, flash memories, or forms of electrically programmable memories (EPROM) or EEPROM. In some examples, storage device 84 is used to store data indicative of instructions for execution by processing circuitry 80. Storage device 84 may be used by software or applications running on external device 12 to temporarily store information during program execution.

Storage device 84 may also store sensed ECG signal(s) 83 which external device 12 may receive from IMD 10. Storage device 84 may also store processed ECG signal(s) 87. For example, communication circuitry 82 may receive a sensed ECG signal from IMD 10 and store the sensed ECG signal in sensed ECG signal(s) 83. Processing circuitry 80 may retrieve the sensed ECG signal from sensed ECG signal(s) 83 and process the sensed ECG signal. For example, processing circuitry may remove low frequency noise, remove high frequency noise, normalize R-wave(s), and/or amplify P-wave(s) when processing the sensed ECG signal. Processing circuitry 80 may store the processed ECG signal in processed ECG signal(s) 87.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., data corresponding to sensed ECG signals, type(s) of suspected cardiac episode(s), cardiac flow rate, optical sensor signals, an accelerometer signal, and/or other collected data) to external device 12. In turn, external device 12 may receive the collected data from IMD 10 and store the collected data in storage device 84. Additionally, or alternatively, processing circuitry 80 may export instructions to IMD 10 requesting IMD 10 to update one or more operational parameters of IMD 10.

A user, such as a clinician, patient 4, or a caregiver, may interact with external device 12 through user interface 86. User interface 86 includes a display (not shown), such as an LCD or LED display or other type of screen, with which processing circuitry 80 may present information related to IMD 10 (e.g., processed ECG signals). In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In some examples, user input may include a selection of a preferred level or strength of processing to be performed by processing circuitry 80 on a sensed ECG signal. In some examples, processing circuitry 80 may store such selection(s) in custom setting(s) 89. When processing circuitry 80 processes a sensed ECG signal, processing circuitry 80 may use the selection(s) to affect a level or strength of the processing.

In some examples, processing circuitry 80 may be configured to tailor the level or strength of processing to a particular clinician. In such examples, processing circuitry 80 may present to the particular clinician a set of example processed ECG signals having different levels or strengths of processing and query the clinician via user interface 86 for a rating of each ECG signal of the set of example processed ECG signals, for example, via user interface 86. The clinician may provide the ratings via user interface 86. Processing circuitry 80 may determine the clinician's preferences based on the provided ratings and store the clinician's preferences in custom setting(s) 89, along with information identifying the clinician. In this manner, the next time processing circuitry 80 processes a sensed ECG signal for the particular clinician for which external device 12 is aware of the identity of the particular clinician (e.g., the clinician may identify themselves by logging into external device 12), processing circuitry 80 may use the custom setting(s) associated with the particular clinician when processing the sensed ECG.

In some examples, processing circuitry 80 may be configured to tailor the level or strength of processing to a particular patient. In such examples, processing circuitry 80 may present to a clinician a set of example processed ECG signals having different levels or strengths of processing, the set of example processed ECG signals all being associated with patient 4, for example. Processing circuitry 80 may query the clinician for a rating of each ECG signal of the set of example processed ECG signals, for example, via user interface 86. The clinician may provide the ratings via user interface 86. Processing circuitry 80 may determine the preferences relating to patient 4 based on the provided ratings and store the preferences in custom setting(s) 89, along with information identifying the patient. In this manner, the next time processing circuitry 80 processes a sensed ECG signal for patient 4 for which external device 12 is aware of the identity of patient 4 (e.g., IMD 10 may include an identifier that IMD 10 transmits to external device 12 which is unique to IMD 10 and thus, unique to patient 4), processing circuitry 80 may use the custom setting(s) associated with patient 4 when processing the sensed ECG.

While three separate customizations are described above, in some examples, any combination of such customizations may be used by processing circuitry 80. For example, processing circuitry 80 may average, use a highest strength processing, use a lowest strength processing, or the like, to combine customizations.

In some examples, user interface 86 also includes audio circuitry for providing audible notifications, instructions or other sounds to patient 4, receiving voice commands from patient 4, or both. Storage device 84 may include instructions for operating user interface 86 and for managing power source 88.

Power source 88 is configured to deliver operating power to the components of external device 12. Power source 88 may include a battery and a power generation circuit to produce the operating power. In some examples, the battery is rechargeable to allow extended operation. Recharging may be accomplished by electrically coupling power source 88 to a cradle or plug that is connected to an alternating current (AC) outlet. In addition, recharging may be accomplished through proximal inductive interaction between an external charger and an inductive charging coil within external device 12. In other examples, traditional batteries (e.g., nickel cadmium or lithium ion batteries) may be used. In addition, external device 12 may be directly coupled to an alternating current outlet to operate.

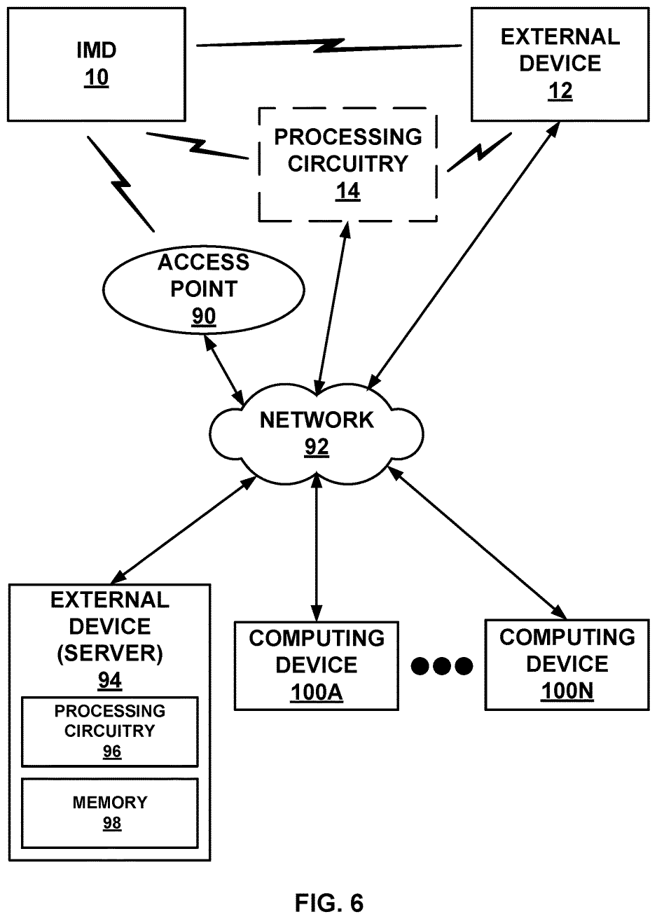

FIG. 6 is a block diagram illustrating an example system that includes an access point 90, a network 92, external computing devices, such as a server 94, and one or more other computing devices 100A-100N, which may be coupled to IMD 10, external device 12, and processing circuitry 14 via network 92, in accordance with one or more techniques described herein. In this example, IMD 10 may use communication circuitry 54 to communicate with external device 12 via a first wireless connection, and to communication with an access point 90 via a second wireless connection. In the example of FIG. 6, access point 90, external device 12, server 94, and computing devices 100A-100N are interconnected and may communicate with each other through network 92.

Access point 90 may include a device that connects to network 92 via any of a variety of connections, such as telephone dial-up, digital subscriber line (DSL), fiber optic, or cable modem connections. In other examples, access point 90 may be coupled to network 92 through different forms of connections, including wired or wireless connections. In some examples, access point 90 may be a user device, such as a tablet or smartphone, that may be co-located with the patient. As discussed above, IMD 10 may be configured to transmit data, such as sensed ECG signals, types of suspected cardiac episodes, optical sensor signals, an accelerometer signal, or other data collected by IMD 10 to external device 12. In addition, access point 90 may interrogate IMD 10, such as periodically or in response to a command from the patient or network 92, in order to retrieve parameter values determined by processing circuitry 50 of IMD 10, or other operational or patient data from IMD 10. Access point 90 may then communicate the retrieved data to server 94 via network 92.

In some cases, server 94 may be configured to provide a secure storage site for data that has been collected from IMD 10, and/or external device 12, such as sensed ECG signal(s) 83, processed ECG signal(s) 87, custom setting(s) 89, and/or physiological parameters of patient 4. In some cases, server 94 may assemble data in web pages or other documents for viewing by trained professionals, such as clinicians, via computing devices 100A-100N. One or more aspects of the illustrated system of FIG. 6 may be implemented with general network technology and functionality, which may be similar to that provided by the Medtronic CareLink® Network developed by Medtronic plc, of Dublin, Ireland.

Server 94 may include processing circuitry 96. Processing circuitry 96 may include fixed function circuitry and/or programmable processing circuitry. Processing circuitry 96 may include any one or more of a microprocessor, a controller, a DSP, an ASIC, an FPGA, or equivalent discrete or analog logic circuitry. In some examples, processing circuitry 96 may include multiple components, such as any combination of one or more microprocessors, one or more controllers, one or more DSPs, one or more ASICs, or one or more FPGAs, as well as other discrete or integrated logic circuitry. The functions attributed to processing circuitry 96 herein may be embodied as software, firmware, hardware or any combination thereof. In some examples, processing circuitry 96 may perform one or more techniques described herein.

Server 94 may include memory 98. Memory 98 includes computer-readable instructions that, when executed by processing circuitry 96, cause IMD 10 and processing circuitry 96 to perform various functions attributed to IMD 10 and processing circuitry 96 herein. Memory 98 may include any volatile, non-volatile, magnetic, optical, or electrical media, such as RAM, ROM, NVRAM, EEPROM, flash memory, or any other digital media. In some examples, server 94 may be configured to perform the signal modification techniques of this disclosure in the manner (or in a similar manner) as described herein with respect to external device 12.

In some examples, one or more of computing devices 100A-100N (e.g., device 100A) may be a tablet or other smart device located with a clinician, by which the clinician may program, receive alerts from, and/or interrogate IMD 10. For example, the clinician may access sensed ECG signal(s) 83, processed ECG signal(s) 87 or other data corresponding to physiological parameters of patient 4 determined by IMD 10, external device 12, processing circuitry 14, and/or server 94 through device 100A, such as when patient 4 is in between clinician visits or when IMD 10 detects a potential cardiac episode, to diagnose a cardiac episode. In some examples, the clinician may enter instructions for a medical intervention for patient 4 into an app in device 100A, such as based on a status of a patient condition determined by IMD 10, external device 12, processing circuitry 14, or any combination thereof, or based on other patient data known to the clinician. Device 100A then may transmit the instructions for medical intervention to another of computing devices 100A-100N (e.g., device 100B) located with patient 4 or a caregiver of patient 4. For example, such instructions for medical intervention may include an instruction to change a drug dosage, timing, or selection, to schedule a visit with the clinician, to take their fluid medication, or to seek medical attention. In further examples, device 100B may generate an indication for output, such as an alert to patient 4 based on a diagnosis of a potential cardiac episode of patient 4, which may enable patient 4 proactively to seek medical attention prior to receiving instructions for a medical intervention. In this manner, patient 4 may be empowered to take action, as needed, to address their medical status, which may help improve clinical outcomes for patient 4.

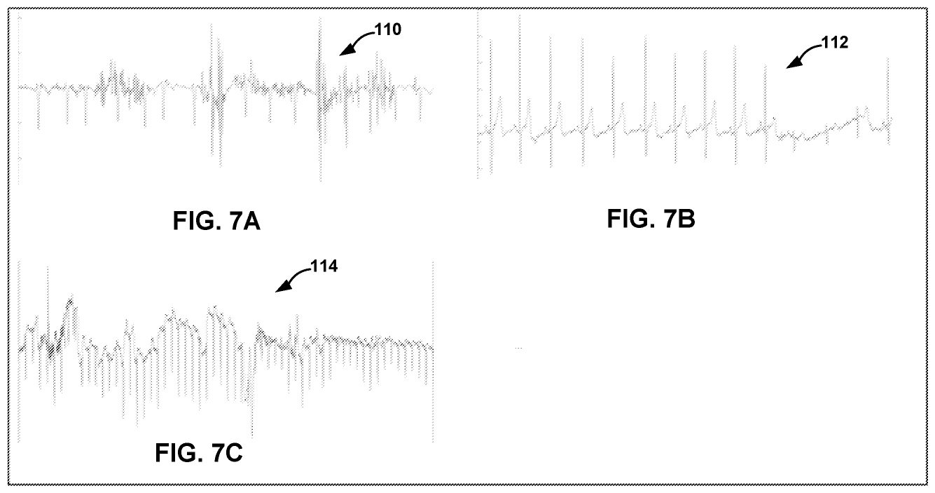

FIGS. 7A-7C are conceptual diagrams illustrating example sensed ECG signals. FIG. 7A includes high frequency noise which may make visual analysis of sensed ECG signal 110 difficult for a clinician. FIG. 7B includes large R-wave amplitude changes which may make visual analysis of sensed ECG signal 112 difficult for a clinician. FIG. 7C includes low frequency noise (e.g., baseline drift) which may make visual analysis of sensed ECG signal 114 difficult for a clinician. As such, the techniques of this disclosure may include processing such sensed ECG signals to improve visualization thereby improving the ability of a clinician to accurately diagnose a potential cardiac episode represented in the sensed ECG signal.

Visualizing R-waves is desirable to interpret true asystole, bradycardia, and/or tachycardia. Visualizing R-waves and the ECG baseline for presence/absence of P-waves and flutter waves is desirable to interpret true atrial fibrillation (AF)/atrial flutter (AFL) and atrioventricular (AV) blocks. Changes in R-wave amplitude levels, high frequency noise, baseline drift artifacts and ECG morphology changes due to the location of the IMD sensing the ECG which may make it difficult to view R-waves and P-waves, especially in ECGs rendered to static views (e.g., pdf reports). For example, high frequency noise in an AF episode may obscure the ECG baseline and may make it difficult to ensure absence of P-waves to adjudicate true AF (FIG. 7A). For example, in ECGs with large R-wave amplitude changes (FIG. 7B) or low-frequency/baseline drift artifact (FIG. 7C), all the R-waves may not be readily visible, making it difficult to adjudicate for the presence of true pause/bradycardia. This is exacerbated when ECG signals are rendered in static views (e.g., pdf reports) for review.

Desirable characteristics for ECG denoising and transformation techniques are now described. Such desirable characteristics may include not altering the underlying ECG morphology (e.g., P, QRS complexes, etc.) in a manner that may lead to arrhythmia misinterpretation (e.g., making a narrow QRS complex broad which may lead to a supraventricular tachycardia being misclassified as a wide-complex tachycardia, removing P-waves, changing a QRS onset and/or a T-wave onset for QT determination applications). It may be acceptable, though, to change some characteristics (such as R-wave and T-wave height) based on arrhythmia context (e.g., pause, AF, tachycardia, bradycardia, AT interpretation, etc.). Desirable characteristics may also include not introducing artifacts that may lead to arrhythmia misinterpretation (e.g., introducing very low frequency baseline artifacts which may incorrectly be interpreted as flutter waves). Desirable characteristics may also include denoising more where noise is present in the sensed ECG signal, and not denoising much where there is little noise in the sensed ECG signal. Desirable characteristics may also include enhancing and/or clarifying ECG characteristics based on a diagnostic context. For example, amplifying and/or normalizing R-waves for pause and/or bradycardia interpretation, denoising and maintaining R-wave morphology for supraventricular tachycardia (SVT) vs VT interpretation, denoising and maintaining R-wave morphology for AF interpretation, and providing on-demand P-wave amplification if a user (e.g., a clinician) wants to zoom and visualize atrial activity and/or P-waves.

Denoising and signal transformation methods for easier visualization by a clinician for arrhythmia interpretation are now described. For example, for denoising a sensed ECG signal received from IMD 10, processing circuitry 80 (FIG. 5) may denoise low-frequency artifact noise, such as baseline drift, and/or may denoise high-frequency noise, such as muscle noise, to generate a processed ECG signal. For example, for transforming a sensed ECG signal received from IMD 10, processing circuitry 80 may normalize an R-wave and/or amplify a P-wave to generate a processed ECG signal. In some examples, processing circuitry 80 may both denoise the ECG signal and transform the ECG signal to generate a processed ECG signal. In this manner, through signal processing techniques described herein, processing circuitry 80 may improve the visualization of a sensed ECG signal for presentation to and analysis by a clinician. As such the techniques of this disclosure may improve the ability of a clinician to correctly diagnose a device sensed cardiac episode.

Low-frequency artifact denoising is now discussed. Techniques such as high pass filtering and linear de-trending may appear to be straightforward solutions to denoise low frequency noise, such as baseline drift artifacts, but such techniques may not be directly applicable to ICM sensed ECGs.

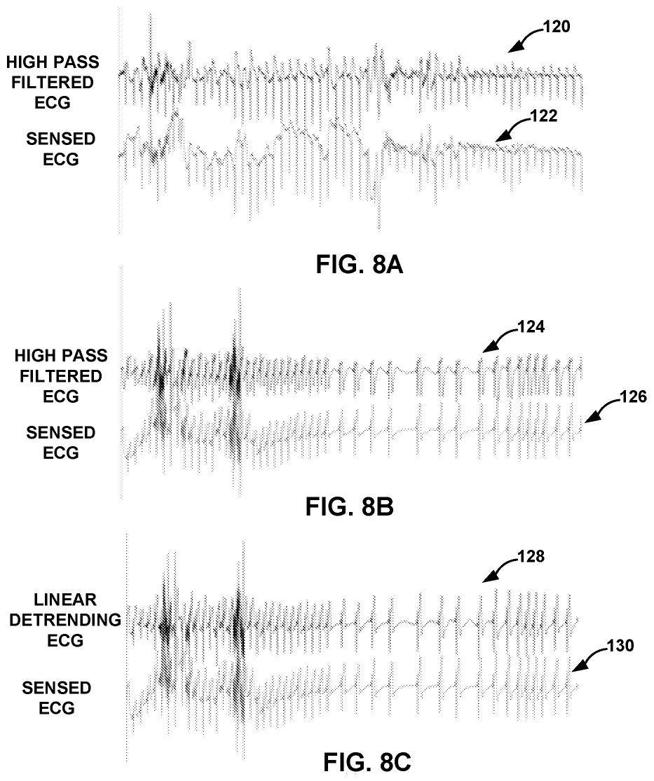

FIGS. 8A-8C are conceptual diagrams of a first high pass filtered ECG, a second high pass filtered ECG, and a linear detrending ECG, respectively. For example, a simple high pass filter may remove baseline drift from sensed ECG signals 122, as shown in high pass filtered ECG signal 120 and from sensed ECG signal 126, as shown in high pass filtered ECG signal 124. However, such high pass filtering may also alter the underlying ECG morphology of an ICM sensed ECG signal, especially the T-wave, as can be seen in FIGS. 8A-8B. Linear detrending may also remove baseline drift from sensed ECG signal 130, as shown in linear detrending ECG signal 128. However, linear detrending may also introduce discontinuities, as is shown in FIG. 8C. Therefore, different techniques to remove low frequency noise may be desirable.

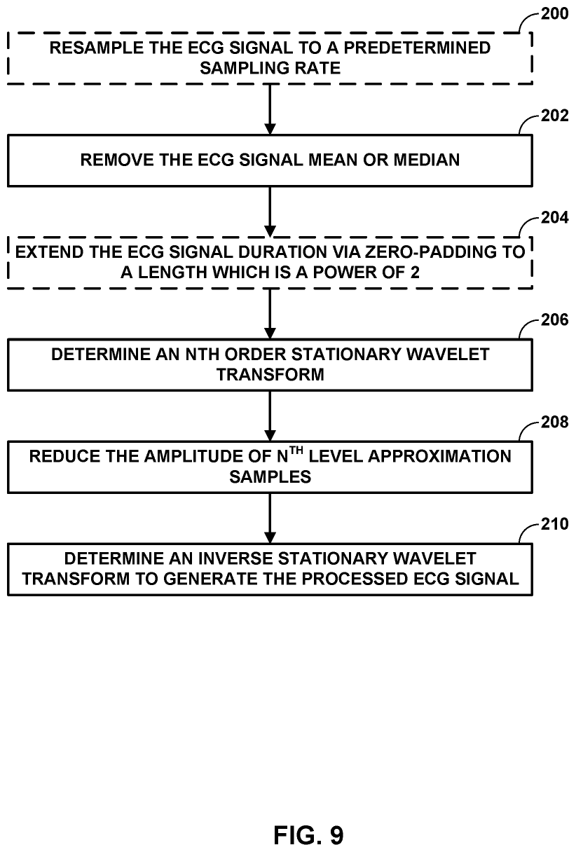

FIG. 9 is a flow diagram illustrating example low frequency artifact denoising techniques according to one or more aspects of this disclosure. While discussed with respect to processing circuitry 80 of FIG. 5, the techniques of FIG. 9 may be implemented by processing circuitry 14 (FIGS. 1 and 6), processing circuitry 50 (FIG. 3), processing circuitry 96 (FIG. 6), or processing circuitry of any device(s) capable of performing such techniques. Processing circuitry 80 may, if desired (this being optional as indicated by the dashed lines in FIG. 9), resample the ECG signal to a pre-specified sampling rate (200). For example, processing circuitry 80 may resample the ECG signal to a rate in the range of 50 to 200 Samples/second, such as 128 Samples/second. Processing circuitry 80 may remove the ECG signal mean or median (202). For example, processing circuitry 80 may determine an ECG signal mean or median and subtract out the ECG signal mean or median from the ECG signal to remove the ECG signal mean or median.

If applicable, processing circuitry 80 may extend the ECG signal duration via zero-padding to a length which is a multiple of power of 2, for example to help with a subsequent stationary wavelet transform (204). For example, processing circuitry 80 may determine whether the ECG signal duration is a multiple of the power of 2, and only apply zero-padding if ECG signal duration is not a multiple of the power of 2. Processing circuitry 80 may keep track of the original ECG signal length, such as by storing the original ECG signal length in storage device 84. Processing circuitry 80 may determine an N^{th} order stationary wavelet transform (206). For example, processing circuitry 80 may determine a 7th order transform with Haar wavelet. The use of a Haar wavelet may be desirable for ICM sensed ECGs, as a Haar wavelet appears to result in better visualization of a resulting ECG than other wavelets, such as a Coiflet wavelet. Processing circuitry 80 may reduce the amplitude of N^{th} level approximation samples (208). For example, processing circuitry 80 may reduce the amplitude of N^{th} level approximation samples to reduce the baseline drift and/or low-frequency artifact of the original sensed ECG signal. For example, processing circuitry may reduce the amplitude of 7th level approximation samples to 0. Processing circuitry 80 may determine an inverse stationary wavelet transform to create a denoised ECG signal (210). This denoised ECG signal may be a re-creation of the first ECG signal without or with reduced baseline drift and/or low-frequency noise. Processing circuitry 80 may retain the original ECG signal length in the denoised ECG signal.

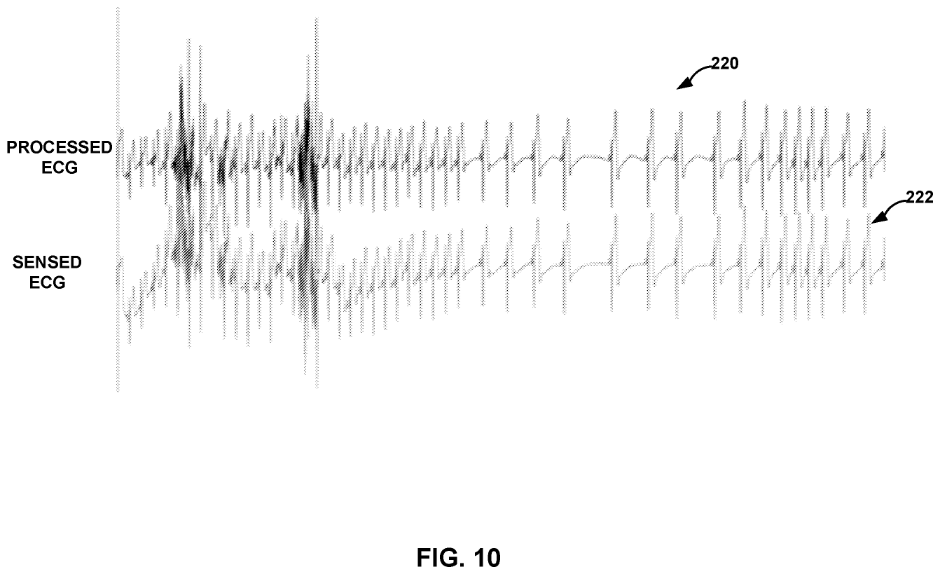

FIG. 10 is a conceptual diagram of an example ECG signal having the baseline drift removed while retaining the underlying P-QRS-T morphologies. By applying the techniques set forth in the example of FIG. 9 to sensed ECG signal 222, processing circuitry 80 may generate a processed ECG signal such as processed ECG signal 220 of the example of FIG. 10.

In some examples, processing circuitry 80 may be configured to provide a user (e.g., clinician) via user interface 86 an ability to control a level or strength of baseline drift and/or low frequency noise removal. For example, user interface 86 may present the user with “aggressive”/“balanced”/“conservative” options for denoising the baseline drift and/or low frequency noise artifact. For example, if the user selects the “aggressive” option, processing circuitry may use a 6^{th} order transformation (which may denoise more of the higher frequencies) and set the amplitude of all the 6^{th} order approximation samples to 0. For example, if the user selects the “balanced” setting, processing circuitry may use a 7^{th} order transformation (which may denoise more of the higher frequencies) and set the amplitude of all the 7^{th} order approximation samples to 0. For example, if the user selects the “conservative” setting, processing circuitry may use an 8^{th} order transformation (which may only denoise very low frequency artifacts) and reduce the amplitude of the 8^{th} order approximation samples' amplitude by 90%. While described herein as presenting three options, other numbers of options are contemplated and other implementations of such options are also contemplated. For example, there may be any number of options.

Muscle noise and/or high-frequency artifact denoising is now discussed. Techniques such as low-pass filtering may appear to be straightforward solutions to remove high-frequency artifacts, but such techniques may not be directly applicable to ICM-sensed ECG signals. For example, a simple low-pass filter may remove high-frequency noise from the ICM-sensed ECG signal, but may also alter the underlying ECG morphology within the ICM-sensed ECG signal. This may be undesirable.

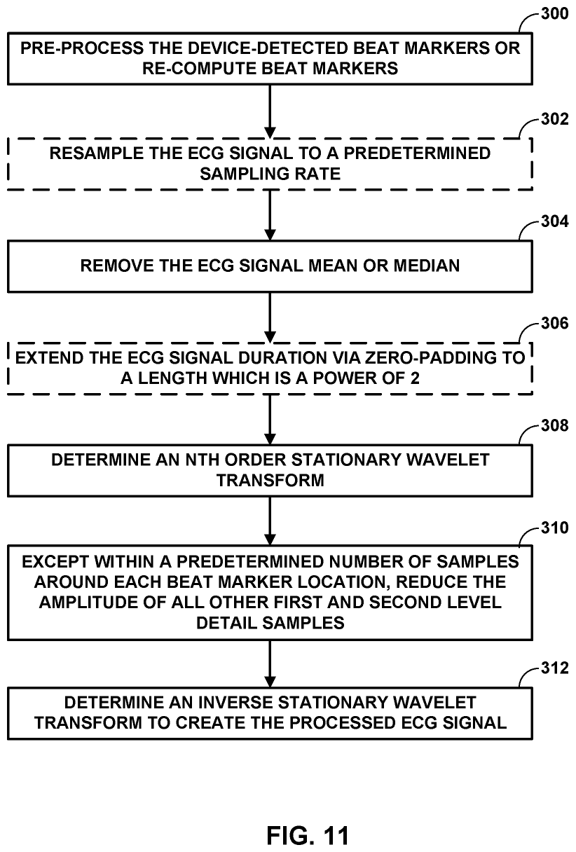

FIG. 11 is a flow diagram illustrating example high-frequency artifact denoising according to one or more aspects of this disclosure. While discussed with respect to processing circuitry 80 of FIG. 5, the techniques of FIG. 11 may be implemented by processing circuitry 14 (FIGS. 1 and 6), processing circuitry 50 (FIG. 3), processing circuitry 96 (FIG. 6), or processing circuitry of any device(s) capable of performing such techniques. Processing circuitry 80 may, based on the type of ECG episode the clinician may want to assess, either pre-process the device-determined beat markers or re-compute beat markers (300). Along with the ICM-sensed ECG signal, processing circuitry 80 may receive ICM-detected beat markers from IMD 10. For example, processing circuitry 80 may, for device-detected AF, AT, bradycardia, patient-activated, routine and/or baseline episodes, pre-process the device-detected beat markers for denoising. For example, processing circuitry 80 may re-compute beat markers for device-detected tachycardia and asystole episodes, because device-detected tachycardia episodes may have QRS oversensing which may lead to subsequent under-denoising of a high frequency artifact and device-detected asystole episodes may have QRS undersensing which can lead to subsequent over-denoising of high-frequencies and QRS distortion, which may be undesirable.

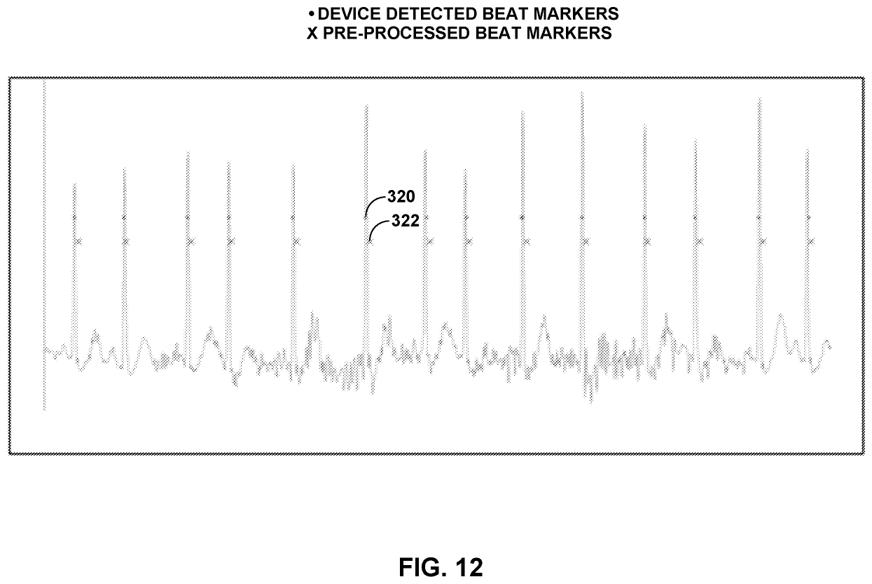

FIG. 12 is a conceptual diagram of an example ECG signal indicating device-detected beat markers and pre-processed beat markers to line up with a QRS maximum slew location according to one or more aspects of this disclosure. For example, processing circuitry 80 may pre-process device-detected beat markers, such as device-detected beat marker 320 to line up the beat marker times to the timing of the highest QRS slew-rate or QRS peak, for example pre-processed beat marker 322. Device-detected beat markers may have limited time resolution and may not line up with a predictable and precise QRS location, which may be desirable to retain the QRS frequencies while denoising high-frequency noise.

Referring back to FIG. 11, if desired (e.g., optionally, as indicated by the dashed lines), processing circuitry 80 may resample the ECG signal to a pre-specified sampling rate (302). For example, processing circuitry 80 may resample the ECG signal to a rate in the range of 50 to 200 Samples/second, such as 128 Samples/second. Processing circuitry 80 may remove an ECG signal mean or median (304). For example, processing circuitry 80 may subtract an ECG signal mean or median from the ECG signal to remove the ECG signal mean or median.

If applicable, processing circuitry 80 may extend the ECG signal duration via zero-padding to a length which is a multiple of power of 2, for example to help with a subsequent stationary wavelet transform (306). For example, processing circuitry 80 may determine whether the ECG signal duration is a multiple of the power of 2, and only apply zero-padding if the ECG signal duration is not a multiple of the power of 2. Processing circuitry 80 may keep track of the original ECG signal length, such as by storing the original ECG signal length in storage device 84. Processing circuitry 80 may determine an N^{th} order stationary wavelet transform (308). For example, processing circuitry 80 may determine a 7^{th} order transform with Haar wavelet. As discussed above, the use of a Haar wavelet may be desirable for ICM-sensed ECGs, as a Haar wavelet appears to result in better visualization of a resulting ECG than other wavelets. In examples where processing circuitry 80 is performing both low-frequency denoising and high-frequency denoising, processing circuitry 80 may use the same transform for both to reduce re-computation and save processing power.

Except within a predetermined number of samples around each beat maker location (e.g., beat markers identified in 300), processing circuitry 80 may reduce all other first and second level detail samples (310). The first 2 stationary wavelet transform detail levels contain high frequency information and reducing these detail samples may reduce high frequency artifact(s). Retaining the samples around the beat locations is desirable to retain the QRS complex high frequency information during ECG signal reconstruction. Thus, processing circuitry 80 may retain such samples. For example, processing circuitry 80 may retain a number of samples, such as 15 samples (~120 ms) around each beat marker, set the other first level details to 0, and reduce the other second level details by 99%. Processing circuitry 80 may determine an inverse stationary wavelet transform to create a processed ECG signal (312). This processed ECG signal may be a re-creation of the original ECG signal without or with reduced high-frequency noise, such as muscle noise. Processing circuitry 80 may retain the original signal length in the denoised signal.

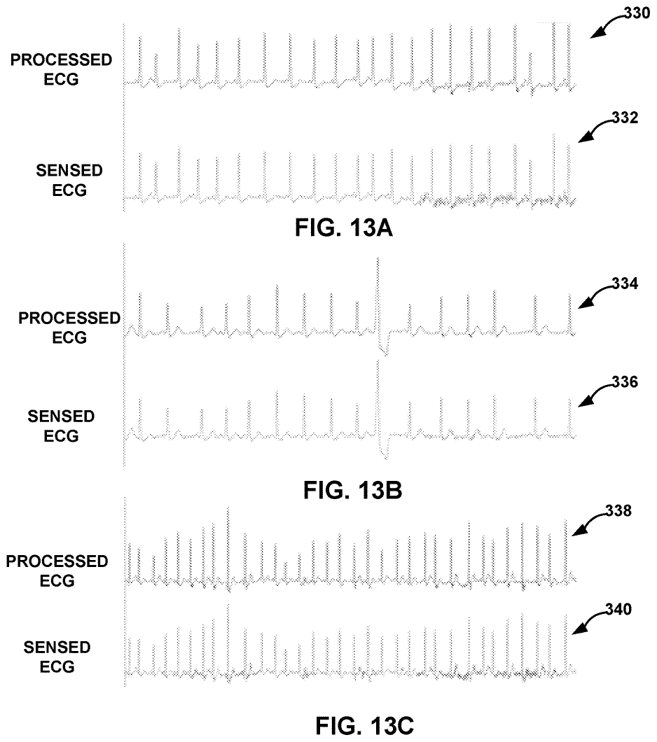

FIGS. 13A-13C are conceptual diagrams illustrating example processed ECG signals and sensed ECG signals according to one or more aspects of this disclosure. Processing circuitry 80 may remove the high frequency noise from a sensed ECG signal while retaining the underlying P-QRS-T morphologies for normal and PVC beats, using the techniques of FIG. 11. For example, processed ECG signal 330, processed ECG signal 334, and processed ECG signal 338, each retain the QRS morphologies of sensed ECG signal 332, sensed ECG signal 336, and sensed ECG signal 340, respectively, including any premature ventricular contractions (PVCs). Additionally, the baseline appears clearer in the processed ECG signals 330, 334, and 338, than in the corresponding sensed ECG signals 332, 336, and 340, respectively, due to the removal of high frequency noise. As such, processed ECG signals 330, 334, and 338 may be easier for a clinician to analyze than sensed ECG signals 332, 336, and 340, respectively.

In some examples, processing circuitry 80 may be configured to provide a user (e.g., clinician) via user interface 86 an ability to control a level or strength of muscle noise and/or high-frequency noise removal. For example, user interface 86 may present the user with “aggressive”/“balanced”/“conservative” options for denoising the muscle noise and/or high-frequency noise. For example, if the user selects the “aggressive” option, processing circuitry 80 may set all the 1st and 2nd level detail samples to 0 (irrespective of beat location). If the user selects the “balanced” option, processing circuitry 80 may retain a number of samples, such as 15 samples (~120 ms) around each beat, set the first level details to 0, and reduce the second level details by 99%. If the user selects the “conservative” option, processing circuitry may reduce the 1st level detail samples' amplitude by 90% and retain samples 200 ms around each beat. While described herein as presenting three options, other numbers of options are contemplated and other implementations of such options are also contemplated. For example, there may be any number of options.

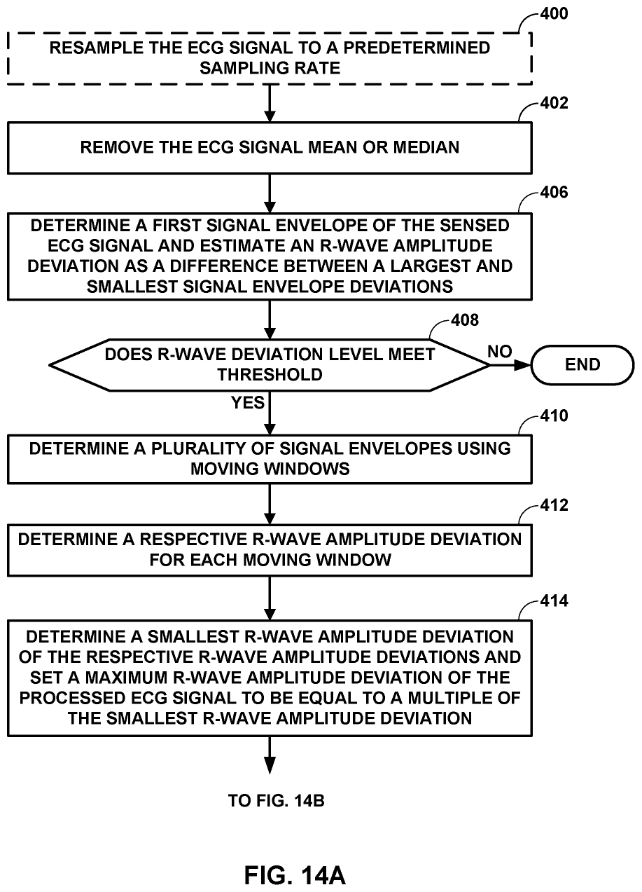

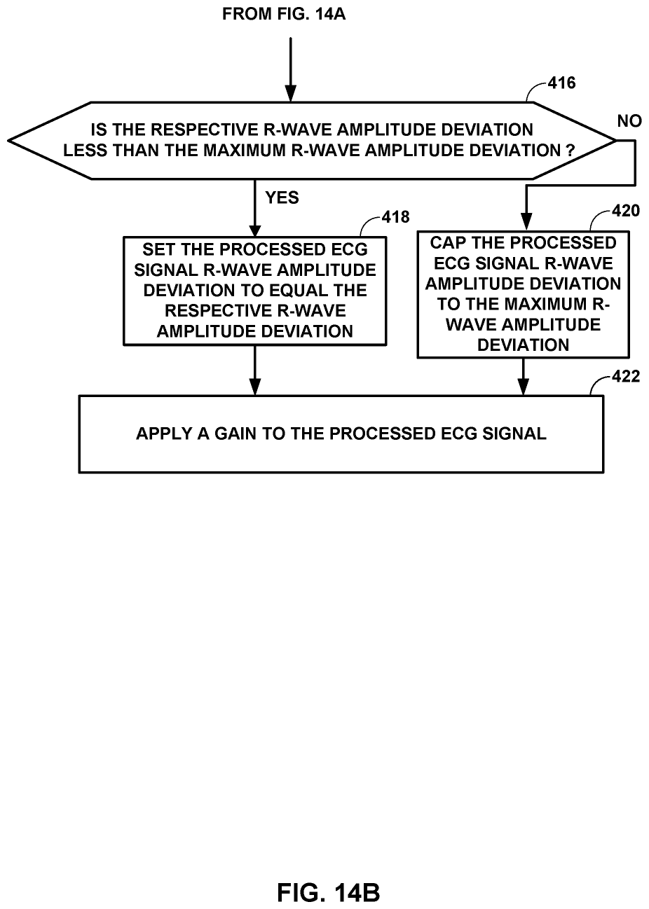

FIGS. 14A-14B are flow diagrams illustrating example R-wave amplitude normalization techniques according to one or more aspects of this disclosure. While discussed with respect to processing circuitry 80 of FIG. 5, the techniques of FIGS. 14A-14B may be implemented by processing circuitry 14 (FIGS. 1 and 6), processing circuitry 50 (FIG. 3), processing circuitry 96 (FIG. 6), or processing circuitry of any device(s) capable of performing such techniques. If desired (e.g., optionally, as indicated by the dashed lines), processing circuitry 80 may resample the ECG signal to a pre-specified sampling rate (400). For example, processing circuitry 80 may resample the ECG signal to a rate in the range of 50 to 200 Samples/second, such as 128 Samples/second. Processing circuitry 80 may remove an ECG signal mean or median (402). For example, processing circuitry 80 may subtract an ECG signal mean or median from the ECG signal to remove the ECG signal mean or median.

Processing circuitry 80 may determine a signal envelope and determine an ECG signal R-wave amplitude deviation as the difference between the largest and smallest envelope deviations (406). For example, processing circuitry 80 may subtract the smallest envelope deviation from the largest envelope deviation. Processing circuitry 80 may then compare the R-wave amplitude deviation level to a deviation threshold (408). If the deviation threshold is not met (the “NO” path from box 408), processing circuitry 80 may end the R-wave normalization techniques and refrain from normalizing the ECG signal. If the deviation threshold is met (the “YES” path from box 408), processing circuitry 80 may apply R-wave normalization. The deviation threshold may be said to be met if the R-wave amplitude deviation level exceeds the deviation threshold is exceeded, if the R-wave amplitude deviation level is equal to the deviation threshold, or both. Thus, processing circuitry 80 applies normalization in cases where the R-wave amplitude deviation level is relatively large.

Processing circuitry 80 may determine a plurality of signal envelopes using moving windows (410). In some examples, the moving window size may be large enough to capture only the R-wave amplitude level (and not the baseline deviation) and small enough such that the moving window captures changes in the R-wave amplitude level over the ECG signal duration. For example, processing circuitry 80 may use an 8-second long moving window having a 1-second movement (e.g., the moving window moves every second). In some examples, processing circuitry 80 may use the 99^{th} and 1^{st} percentile of samples in each window and smooth the values across the moving windows to determine the plurality of envelopes.

Processing circuitry 80 may determine a respective R-wave amplitude deviation of each moving window (412). For example, processing circuitry 80 may determine the respective deviations as the difference between the largest and smallest envelope deviations within each moving window.

Processing circuitry 80 may determine the smallest R-wave amplitude deviation of the respective R-wave amplitude deviations (e.g., the smallest deviation over the ECG signal duration) and set a maximum R-wave amplitude deviation of the processed ECG signal to be equal to a multiple of the smallest deviation (414). For example, processing circuitry 80 may set normalized maximum R-wave amplitude deviation as twice the smallest R-wave amplitude deviation. Processing circuitry 80 may compare each respective R-wave amplitude deviation to the maximum R-wave amplitude deviation (416). If the respective R-wave amplitude deviation is less than the maximum R-wave amplitude deviation (the “YES” path from box 416), processing circuitry 80 may set the processed ECG signal R-wave amplitude deviation to equal the respective R-wave amplitude deviation (418). If the respective R-wave amplitude deviation is equal to or greater than the maximum R-wave amplitude deviation (the “NO” path from box 416), processing circuitry 80 may cap the processed ECG signal R-wave amplitude deviation to the maximum R-wave amplitude deviation (420).

Processing circuitry 80 may apply a gain to the processed the ECG signal (422). For example, the gain may be equal to the processed signal's R-wave amplitude deviation divided by the respective R-wave amplitude deviation. In some examples, processing circuitry 80 may filter the normalization gain to smooth out abrupt changes. Processing circuitry 80 may multiply the original signal by the determined gain to normalize the ECG signal.

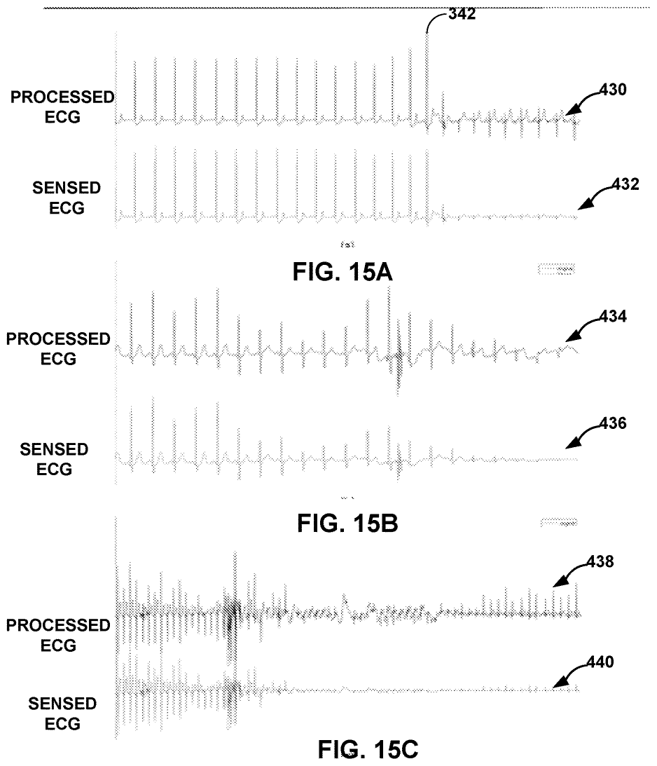

FIGS. 15A-15C are conceptual diagrams illustrating example processed ECG signals and corresponding sensed ECG signals according to one or more aspects of this disclosure. Processing circuitry 80 may make the R-waves more easily visible which may assist a clinician to avoid misdiagnosing a cardiac event in an ECG as a true pause as shown in FIGS. 15A-C by employing the techniques of FIGS. 14A-B. For example, as shown in FIGS. 15A-C, the R-waves (e.g., R-wave 342) are more easily visible in processed ECG signals 430, 434, and 438 than in sensed ECG signals 432, 436, and 440, respectively.

P-wave amplification is now discussed. It may be desirable to enhance/clarify certain ECG characteristics based on diagnostic context, e.g., easily visualize P-waves to confirm or rule out AF. A generic zoom functionality may be insufficient to more easily visualize P-waves because such functionality may make it difficult to visualize R-waves and P-waves at the same time.

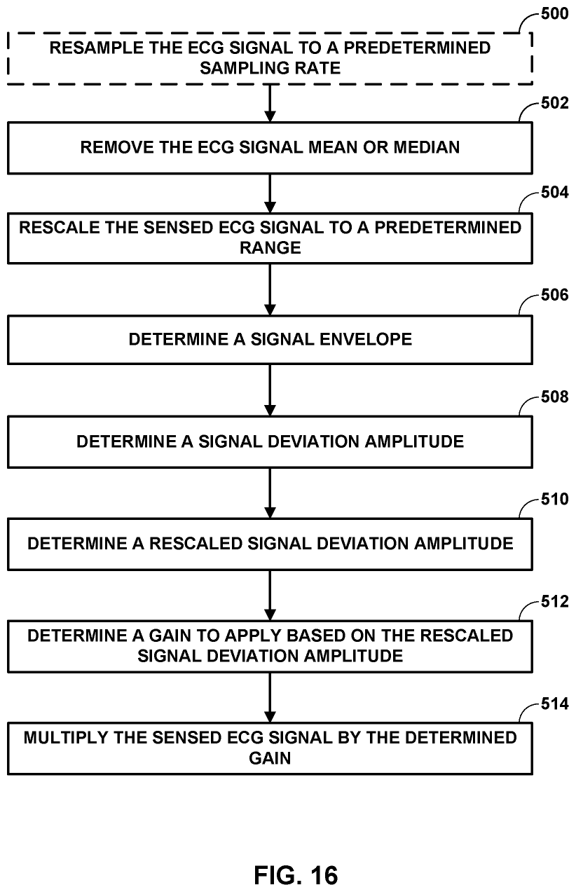

FIG. 16 is a flow diagram illustrating example P-wave amplification techniques according to one or more aspects of this disclosure. While discussed with respect to processing circuitry 80 of FIG. 5, the techniques of FIG. 16 may be implemented by processing circuitry 14 (FIGS. 1 and 6), processing circuitry 50 (FIG. 3), processing circuitry 96 (FIG. 6), or processing circuitry of any device(s) capable of performing such techniques. According to the techniques of this disclosure, processing circuitry may amplify P-waves in such a manner that enhances the P height to R height ratio. If desired (e.g., optionally, as indicated by the dashed lines), processing circuitry 80 may resample the ECG signal to a pre-specified sampling rate (500). For example, processing circuitry 80 may resample the ECG signal to a rate in the range of 50 to 200 Samples/second, such as 128 Samples/second. Processing circuitry 80 may remove an ECG signal mean or median (502). For example, processing circuitry 80 may subtract an ECG signal mean or median from the ECG signal to remove the ECG signal mean or median.

Processing circuitry 80 may rescale the sensed ECG signal to a standardized range (504). This is desirable to apply the subsequent amplification method irrespective of the input signal range. For example, processing circuitry 80 may rescale the entirety of the sensed ECG signal from the ECG signal's [0.1^{st} to 99.9^{th}] percentile to [0 to 1].

Processing circuitry 80 may determine a signal envelope (506). For example, processing circuitry 80 may determine the signal envelope using a short-enough moving window to capture the baseline and R-wave amplitude changes and long enough window to be unaffected by intermittent noise. For example, processing circuitry may use a 62.5 ms window size with a 62.5 ms movement and [0.1^{st}, 99.9^{th}] percentile values for the signal envelope. Processing circuitry 80 may determine a signal deviation amplitude (508). For example, processing circuitry 80 may subtract the lowest amplitude value of the moving window from the highest amplitude value of the moving window (e.g., deviation=highest amplitude value-lowest amplitude value of the moving window). Processing circuitry 80 may determine the rescaled signal deviation amplitude (510). For example, processing circuitry 80 may use a generalized logistic function such as:

Rescaled signal deviation amplitude=A+(K−A)/(C+Qe^{-B*(original signal deviation)})^{1/v}

The generalized logistic function parameters may be chosen to amplify the smaller deviations (baseline/P-wave amplitudes) and cap the larger deviations (R-wave amplitude levels). For examples, the generalized logic function parameters may include A=0, K=1, C=1, B=10, Q=0.5, v=0.1 or other values.

Processing circuitry 80 may determine a gain to apply based on the rescaled signal deviation amplitude (512). For example, the gain may be an inverse of the rescaled amplitude deviation level, e.g., a gain that is high for lower amplitudes (P-waves, flutter waves) and is capped at 1 for the higher amplitudes (R-waves) to amplify the P-waves and flutter waves and keep R-wave amplitudes at the same level. For example, the gain may be determined as Gain=0.8/(0.8+the rescaled signal deviation amplitude).

Processing circuitry 80 may multiply the sensed ECG signal by the determined gain (514). In some examples, processing circuitry 80 may amplify the P-waves by a factor of 2. While it is possible to further amplify the P-waves, it is not desirable because the underlying ECG morphology may also be highly distorted.

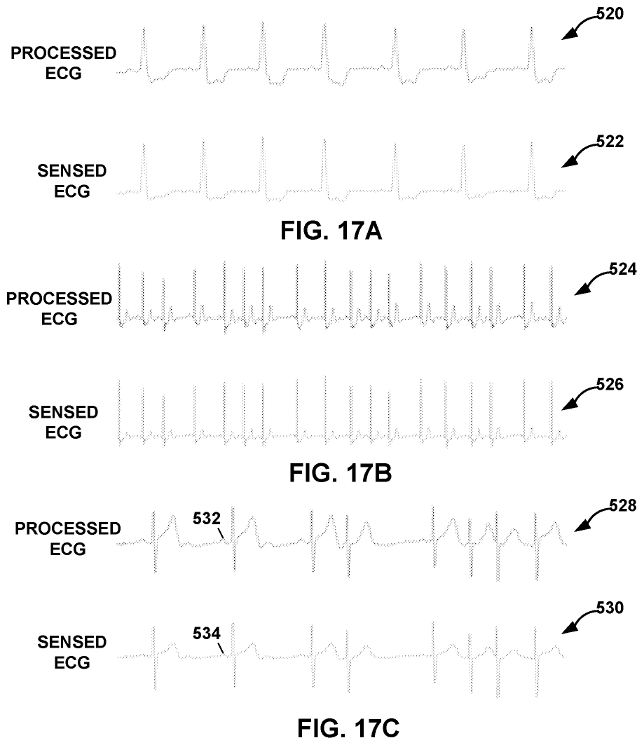

FIGS. 17A-17C are conceptual diagrams illustrating example processed ECGs and corresponding sensed ECGs according to one or more aspects of this disclosure. For example, processing circuitry 80 may make the P-waves more easily visible as shown in FIGS. 17A-C by employing the techniques of FIG. 16. As can be seen in FIGS. 17A-C, the P-waves are more easily visible in processed ECG signals 520, 524, and 528 than in corresponding sensed ECG signals 522, 526, and 530, respectively. For example, P-wave 532 is more easily visible than P-wave 534.

Combining signal denoising and transformation techniques is now discussed. It may be desirable to combine the individual denoising and transformation techniques detailed above because (i) multiple artifacts may be present in a sensed ECG of a device-detected cardiac episode and (ii) depending on the type of device-detected cardiac episode, different ECG features are more diagnostically relevant. For example, a clinician may desire R-wave visibility for diagnosing a potential pause episode. The individual signal denoising and transformation parameters and the order of application may be carefully chosen since the overall objective is to remove artifacts while maintaining the underlying ECG morphology. For example, for visualizing device-detected AF episodes, processing circuitry 80 may remove high-frequency artifacts followed by baseline drift/low-frequency artifacts and R-wave envelope normalization. In such cases, processing circuitry 80 may apply P-wave amplification per a user selection via user interface 86. In some examples, as a default, processing circuitry 80 does not apply or refrains from applying P-wave amplification.

For visualizing device-detected pause, bradycardia, tachycardia, AT, patient-activated and baseline ECGs, processing circuitry 80 may remove baseline drift/low-frequency artifacts followed by high frequency artifacts, and R-wave envelope normalization. For AT episodes, processing circuitry 80 may apply P-wave amplification per a user selection via user interface 86. In some examples, as a default, processing circuitry 80 does not apply or refrains from applying P-wave amplification.

In some examples, processing circuitry 80 may be configured to personalize the denoising and signal transformation for each reviewer. For example, rather than use the parameters set forth above, which may be based on preferences of a large population of clinicians, each clinician or group of clinicians may be presented a set of representative ECG waveforms (e.g., ICM and specific arrhythmia episode types) applied with different combinations of denoising and signal transformation parameter via user interface 86 to obtain user feedback as a rating (e.g., 0-10) on (i) how clean the processed signal looks and (ii) how easy is the processed signal to review for arrhythmias. Processing circuitry may then map (e.g., with machine learning techniques) the combination of parameters to user ratings to determine an optimal set of denoising and transformation operations individualized to each reviewer or group of reviewers. This is shown in the tables below

TABLE 1
Techniques Applied

| High-freq | Low-freq | R-wave | P-wave | |
|---|---|---|---|---|
| filter | filter | Normalize | Amplify | ECG |
| Aggressive | No | No | No | Processed |
| | | | | ECG1 |
| No | Conservative | No | No | Processed |
| | | | | ECG2 |
| . . . | . . . | . . . | . . . | . . . |
| Balanced | Balanced | No | Aggressive | Processed |
| | | | | ECGK |
| . . . | . . . | . . . | . . . | . . . |

TABLE 2
User Feedback

| | | Arrythmia |
|---|---|---|
| ECG | Clean Signal | review ease |
| Processed ECG1 | 6 | 5 |
| Processed ECG2 | 2 | 3 |
| . . . | . . . | . . . |
| Processed ECGK | 8 | 4 |
| . . . | . . . | . . . |

For example, a k-means clustering model may be used having clusters for each potential level or strength of processing. Each rated processing level or strength may be associated with a vector that includes variables for type of device-detected cardiac episode, desired strength of processing, how clean the sensed ECG signal may be, how easy the sensed ECG signal is to review, how clean the processed ECG signal may be, how easy the processed ECG signal is to review, and/or the like. The location of the vector in a given one of the clusters may be indicative of the strength of processing to be applied by, for example, processing circuitry 80. Other potential machine learning techniques include Naïve Bayes, k-nearest neighbors, random forest, support vector machines, neural networks, linear regression, logistic regression, etc.

Another example includes personalizing the denoising and signal transformation for each patient. The type and level of noise and ECG morphology can vary in patients due to the patient's underlying arrhythmia conditions and device placement. To individualize the ECG denoising and transformation for each patient, processing circuitry 80 may select the first N episodes from a patient, apply different combinations of signal denoising and transformation, and obtain user feedback via user interface 86 as to a rating of (i) how clean the processed signal looks and (ii) how easy is the processed signal to review for arrhythmias. Processing circuitry 80 may (e.g., with machine learning techniques as described above) the combination of parameters to user ratings to determine an optimal set of denoising and transformation operations individualized for each patient.

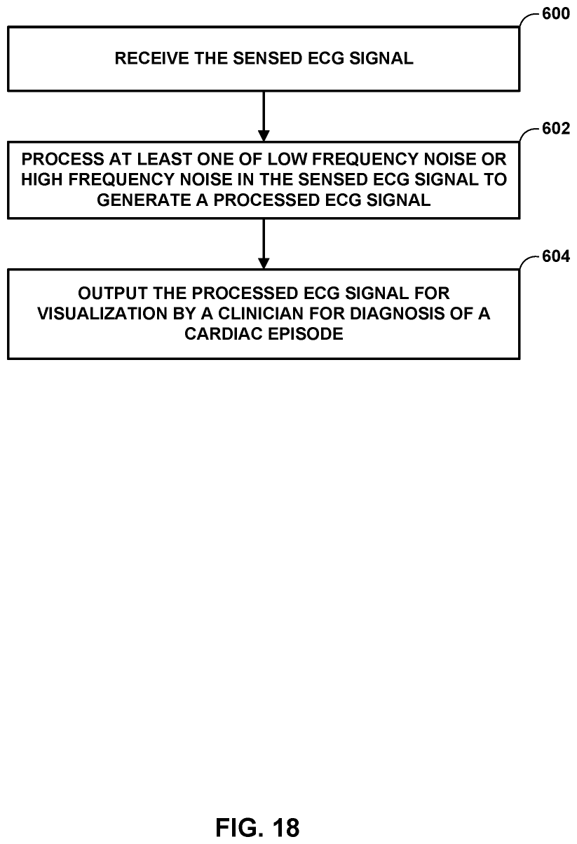

FIG. 18 is a flow diagram of example visualization techniques according to one or more aspects of this disclosure. While discussed with respect to processing circuitry 80 of FIG. 5, the techniques of FIG. 18 may be implemented by processing circuitry 14 (FIGS. 1 and 6), processing circuitry 50 (FIG. 3), processing circuitry 96 (FIG. 6), or processing circuitry of any device(s) capable of performing such techniques.

Processing circuitry 80 may receive the sensed ECG signal (600). For example, communication circuitry 54 of IMD 10 (FIG. 1) may transmit the sensed ECG signal to communication circuitry 82 of external device 12 (FIG. 5) and processing circuitry 80 may receive the sensed ECG signal from communication circuitry 82.

Processing circuitry 80 may process at least one of low frequency noise or high frequency noise in the sensed ECG signal to generate a processed ECG signal (602). For example, processing circuitry 80 may employ the techniques of at least one of FIG. 9 or FIG. 11 to generate the processed ECG signal. For example, processing circuitry 80 may mitigate the at least one of the low frequency noise or the high frequency noise. For example, to mitigate means to reduce the amplitude of, reduce the presence or occurrence of (e.g., an amount of time in an ECG signal during which such noise is present), remove, improve a signal-to-noise ratio, and/or the like. The sensed ECG signal may include a plurality of waves and the processing of the ECG signal may preserve a morphology of the waves. For example, the morphology of the processed ECG signal may be substantially similar to the morphology of the sensed ECG signal. In some examples, a substantially similar morphology may include peaks of one or more different waves (e.g., an R-wave, a P-wave, a T-wave, or the like) of the processed ECG being in the same position (e.g., along the x-axis of an ECG waveform) as in the sensed ECG signal. In some examples, a substantially similar morphology may include peaks of one or more different waves (e.g., an R-wave, a P-wave, a T-wave, or the like) of the processed ECG being within less than 5% of the same position (as determined by an interval between consecutive waves of a same type, e.g., consecutive R-waves, consecutive P-waves, consecutive T-waves, or the like) as in the sensed ECG signal. As part of processing the at least one of low frequency noise or high frequency noise in the sensed ECG signal, processing circuitry 80 may determine one or more wavelet transforms to the sensed ECG signal.

Processing circuitry 80 may output the processed ECG signal with the at least one of the low frequency noise or the high frequency noise mitigated and the morphology of the waves preserved for visualization by a clinician for diagnosis of a cardiac episode (604). For example, processing circuitry 80 may output the processed ECG signal to user interface 86, a display device (not shown), a printer (not shown), or the like.

In some examples, to process the at least one of the low frequency noise or the high frequency noise including determining the one more wavelet transforms, processing circuitry 80 may determine a wavelet transform of the ECG signal; process the wavelet transform; and determine an inverse wavelet transform of the processed wavelet transform to generate the processed ECG signal. In some examples, processing circuitry 80 may determine that one or more visualization criterion associated with the sensed ECG signal are met. In such examples, processing circuitry 80 may process the at least one of the low frequency noise or the high frequency noise in the sensed ECG signal to generate the processed ECG signal is based on the one or more visualization criterion associated with the sensed ECG signal being met. In some examples, the one or more visualization criterion include at least one of an amplitude of the low frequency noise meeting a threshold or an amplitude of the high frequency noise meeting a threshold.

In some examples, the wavelet transform may be an Nth order stationary wavelet transform. In some examples, as part of processing the low frequency noise, processing circuitry 80 may remove an ECG signal mean or median from the sensed ECG signal Processing circuitry 80 may reduce an amplitude of Nth level approximation samples. In some examples, wherein the Nth order stationary wavelet transform includes a 7th order transform using a Haar wavelet.

In some examples, the wavelet transform is an Nth order stationary wavelet transform. In some examples, processing circuitry 80 may, as part of processing the high frequency noise, pre-process device-determined beat markers or re-compute beat markers. Processing circuitry 80 may remove an ECG signal mean or median from the sensed ECG signal. Processing circuitry 80 may, except within a predetermined number of samples around each beat marker location, reduce an amplitude of all other first and second level detail samples. In some examples, the Nth order stationary wavelet transform comprises a 7th order transform using a Haar wavelet.

In some examples, processing circuitry 80 may receive, from a clinician via a user interface, an indication of a selected level of processing of the at least one of the low frequency noise or the high frequency noise in the sensed ECG signal, wherein the processing the at least one of low frequency noise or high frequency noise in the sensed ECG signal is based, at least in part, on the indication.

In some examples, processing circuitry 80 may receive an indication of a type of the device-detected cardiac episode, wherein the processing the at least one of low frequency noise or high frequency noise in the sensed ECG signal is based, at least in part, on the indication. In some examples, processing circuitry 80 may at least one of: determine whether the indication is of a device-detected atrial fibrillation (AF) episode, wherein if the indication is of the device-detected AF episode, the processing circuitry is configured to process the high frequency noise and process the low frequency noise after processing the high frequency noise; or determine whether the indication is of a device-detected pause, bradycardia, tachycardia, or atrial tachycardia, wherein if the indication is of the device-detected pause, bradycardia, tachycardia, or atrial tachycardia, the processing circuitry is configured to process the low frequency noise and process the high frequency noise after processing the low frequency noise.

In some examples, processing circuitry 80 may present to a clinician a set of representative processed ECG signals, the set of representative processed ECG signals comprising a plurality of processed ECG signals being processed differently. Processing circuitry 80 may receive clinician visualization ratings for one or more of the plurality of processed ECG signals, wherein the processing of the at least one of low frequency noise or high frequency noise in the sensed ECG signal is based, at least in part, on the clinician visualization ratings.

In some examples, processing circuitry 80 may present to a clinician a set of representative processed ECG signals, the set of representative processed ECG signals comprising a plurality of processed ECG signals of a particular patient being processed differently. Processing circuitry 80 may receive patient visualization ratings from a clinician for one or more of the plurality of processed ECG signals, wherein the processing of the at least one of low frequency noise or high frequency noise in the sensed ECG signal is based, at least in part, on the patient visualization ratings.

In some examples, as part of generating the processed ECG signal, processing circuitry 80 may, at least one of, normalize at least one R-wave amplitude or amplify at least one P-wave amplitude. In some examples, the sensed ECG signal is sensed by an insertable cardiac monitor via a plurality of subcutaneous electrodes.

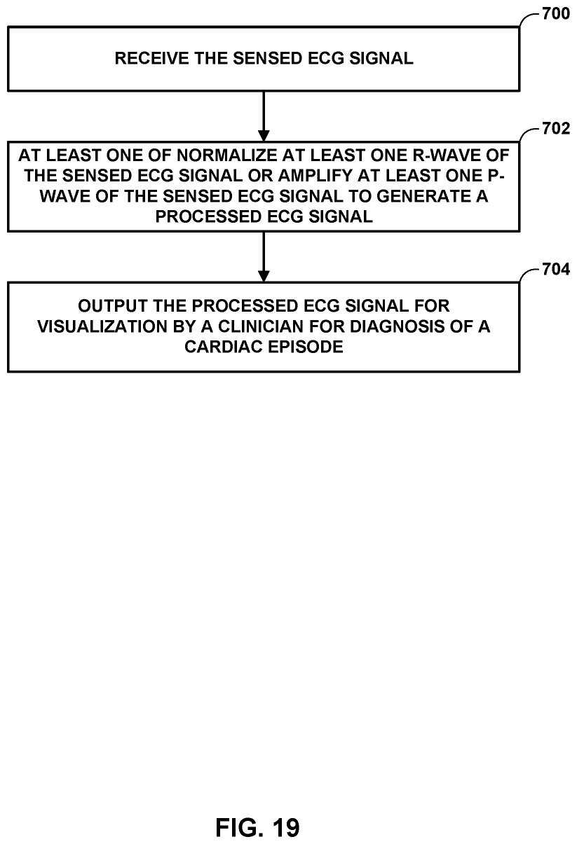

FIG. 19 is another flow diagram of example visualization techniques according to one or more aspects of this disclosure. While discussed with respect to processing circuitry 80 of FIG. 5, the techniques of FIG. 19 may be implemented by processing circuitry 14 (FIGS. 1 and 6), processing circuitry 50 (FIG. 3), processing circuitry 96 (FIG. 6), or processing circuitry of any device(s) capable of performing such techniques.

Processing circuitry 80 may receive the sensed ECG signal (700). For example, communication circuitry 54 of IMD 10 (FIG. 1) may transmit the sensed ECG signal to communication circuitry 82 of external device 12 (FIG. 5) and processing circuitry 80 may receive the sensed ECG signal from communication circuitry 82.

Processing circuitry 80 may, at least one of, normalize at least one R-wave of the sensed ECG signal or amplify at least one P-wave of the sensed ECG signal to generate a processed ECG signal (702). For example, processing circuitry 80 may employ the techniques of at least one of FIGS. 14A-14B or FIG. 16 to the sensed ECG signal to generate the processed ECG signal. For example, processing circuitry 80 may apply a first amplitude gain to the sensed ECG signal, the first amplitude gain based on an R-wave amplitude deviation, and/or apply a second amplitude gain to the sensed ECG signal, the second amplitude gain based on a sensed ECG signal deviation.

Processing circuitry 80 may output the processed ECG signal for visualization by a clinician for diagnosis of a cardiac episode (704). For example, processing circuitry 80 may output the processed ECG signal to user interface 86, a display device (not shown), a printer (not shown), or the like.

In some examples, processing circuitry 80 may determine that one or more visualization criterion associated with the sensed ECG signal are met. In such examples, the at least one of normalizing the at least one R-wave of the sensed ECG signal or amplifying the at least one P-wave of the sensed ECG signal to generate a processed ECG signal is based on the one or more visualization criterion associated with the sensed ECG signal being met. In some examples, the one or more visualization criterion include at least one of an R-wave amplitude deviation level meeting a threshold or an amplitude of a P-wave meeting a threshold.

In some examples, as part of normalizing the at least one R-wave, processing circuitry 80 may remove an ECG signal mean or median from the sensed ECG signal. Processing circuitry 80 may determine a first signal envelope of the sensed ECG signal and determine an R-wave amplitude deviation, the determined ECG signal R-wave amplitude deviation comprising a difference between a largest and a smallest signal envelope deviations. Processing circuitry 80 may determine that the R-wave amplitude deviation meets a threshold. Processing circuitry 80 may determine a plurality of signal envelopes using moving windows. Processing circuitry 80 may determine a respective R-wave amplitude deviation for each moving window. Processing circuitry 80 may determine a smallest R-wave amplitude deviation of the respective R-wave amplitude deviations and set a maximum R-wave amplitude deviation of the processed ECG signal to be equal to a multiple of the smallest R-wave amplitude deviation (e.g., twice the smallest R-wave amplitude deviation. Processing circuitry 80 may determine that a respective R-wave amplitude deviation is less than the maximum R-wave amplitude deviation. Processing circuitry 80 may set an R-wave amplitude deviation of the processed ECG signal to equal the respective R-wave amplitude deviation. Processing circuitry 80 may apply a gain to the processed ECG signal.

In some examples, as part of normalizing the at least one R-wave, processing circuitry 80 may remove an ECG signal mean or median from the sensed ECG signal. Processing circuitry 80 may determine a first signal envelope of the sensed ECG signal and determine an R-wave amplitude deviation, the determined ECG signal R-wave amplitude deviation comprising a difference between a largest and a smallest signal envelope deviations. Processing circuitry 80 may determine that the R-wave amplitude deviation level meets a threshold. Processing circuitry 80 may determine a plurality of signal envelopes using moving windows. Processing circuitry 80 may determine a respective R-wave amplitude deviation for each moving window. Processing circuitry 80 may determine a smallest R-wave amplitude deviation of the respective R-wave amplitude deviations and set a maximum R-wave amplitude deviation of the processed ECG signal to be equal to or less than the smallest R-wave amplitude deviation. Processing circuitry 80 may determine that a respective R-wave amplitude deviation is not less than the maximum R-wave amplitude deviation. Processing circuitry 80 may cap an R-wave amplitude deviation of the processed ECG signal to the maximum R-wave amplitude deviation. Processing circuitry 80 may apply a gain to the processed ECG signal.

In some examples, as part of determining the first signal envelope of the sensed ECG signal, processing circuitry 80 may apply a moving window to the sensed ECG signal, the moving window having an 8 second length and a 1 second movement.

In some examples, as part of amplifying the at least one P-wave, processing circuitry 80 may remove an ECG signal mean or median from the sensed ECG signal. Processing circuitry 80 may rescale the sensed ECG signal to a predetermined range. Processing circuitry 80 may determine a second signal envelope. Processing circuitry 80 may determine a signal deviation amplitude. Processing circuitry 80 may determine a rescaled signal deviation amplitude. Processing circuitry 80 may determine a gain based on the rescaled signal deviation amplitude. Processing circuitry 80 may multiply the sensed ECG signal by the determined gain. In some examples, as part of determining the second signal envelope of the sensed ECG signal, processing circuitry 80 may apply a moving window to the sensed ECG signal, the moving window having a 62.5 ms length and a 62.5 ms movement.

In some examples, as part of determining the rescaled signal deviation amplitude, processing circuitry 80 may apply the formula:

Rescaled signal deviation amplitude=A+(K−A)/(C+Q e^{-B*(original signal deviation)})^{1/v}

where A=0, K=1, C=1, B=10, Q=0.5, and v=0.1.

In some examples, as part of determining the gain, processing circuitry 80 may apply the formula:

Gain=0.8/(0.8+rescaled signal deviation amplitude).

In some examples, processing circuitry 80 may receive an indication of a type of the device-detected cardiac episode, wherein the processing the at least one of low frequency noise or high frequency noise in the sensed ECG signal is based, at least in part, on the indication. In some examples, processing circuitry 80 may determine that the indication is of a device-detected atrial fibrillation (AF) episode, pause, bradycardia, tachycardia, or atrial tachycardia. Based on the determination that the indication is of a device-detected atrial fibrillation (AF) episode, pause, bradycardia, tachycardia, or atrial tachycardia, processing circuitry 80 may normalize the at least one R-wave.

In some examples, processing circuitry 80 may present to a clinician a set of representative processed ECG signals, the set of representative processed ECG signals comprising a plurality of processed ECG signals being processed differently. Processing circuitry 80 may receive clinician visualization ratings for one or more of the plurality of processed ECG signals, wherein the at least one of normalizing of the at least one R-wave or amplifying the at least one P-wave in the sensed ECG signal is based, at least in part, on the clinician visualization ratings.

In some examples, processing circuitry 80 may present to a clinician a set of representative processed ECG signals, the set of representative processed ECG signals comprising a plurality of processed ECG signals of a particular patient being processed differently. Processing circuitry 80 may receive patient visualization ratings from a clinician for one or more of the plurality of processed ECG signals, wherein the at least one of normalizing of the at least one R-wave or amplifying the at least one P-wave in the sensed ECG signal in the sensed ECG signal is based, at least in part, on the patient visualization ratings.

In some examples, as part of generating the processed ECG signal, processing circuitry 80 may process at least one of low frequency noise or high frequency noise in the sensed ECG signal. In some examples, the sensed ECG signal is sensed by an insertable cardiac monitor via a plurality of subcutaneous electrodes.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as clinician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, FRAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

This disclosure includes the following non-limiting examples.

Example 1A. A system for processing a sensed electrocardiogram (ECG) signal to change a visualization of the sensed ECG signal, the system comprising: a memory configured to store the sensed ECG signal; and processing circuitry communicatively coupled to the memory, the processing circuitry being configured to: receive the sensed ECG signal; process at least one of low frequency noise or high frequency noise in the sensed ECG signal to generate a processed ECG signal in which the at least one of the low frequency noise or the high frequency noise is mitigated, wherein the ECG signal includes a plurality of waves, and the processing of the at least one of the low frequency noise or the high frequency noise preserves a morphology of the waves, and wherein as part of processing the at least one of low frequency noise or high frequency noise in the sensed ECG signal, the processing circuitry is configured to determine one or more wavelet transforms to the ECG signal; and output the processed ECG signal with the at least one of the low frequency noise or the high frequency noise mitigated and the morphology of the waves preserved for visualization by a clinician for diagnosis of a cardiac episode.

Example 2A. The system of example 1, wherein to process the at least one of the low frequency noise or the high frequency noise comprising determining the one more wavelet transforms, the processing circuitry is configured to: determine a wavelet transform of the ECG signal; process the wavelet transform; and determine an inverse wavelet transform of the processed wavelet transform to generate the processed ECG signal.

Example 3A. The system of example 1A or example 2A, wherein the processing circuitry is further configured to determine that one or more visualization criterion associated with the sensed ECG signal are met, wherein processing the at least one of the low frequency noise or the high frequency noise in the sensed ECG signal to generate the processed ECG signal is based on the one or more visualization criterion associated with the sensed ECG signal being met.

Example 4A. The system of example 3A, wherein the one or more visualization criterion comprise at least one of an amplitude of the low frequency noise meeting a threshold or an amplitude of the high frequency noise meeting a threshold.

Example 5A. The system of any of examples 1A-4A, wherein the wavelet transform is an Nth order stationary wavelet transform, and wherein the processing circuitry is configured to process the low frequency noise, and as part of processing the low frequency noise, the processing circuitry is configured to: remove an ECG signal mean or median from the sensed ECG signal; and reduce an amplitude of Nth level approximation samples.

Example 6A. The system of example 5A, wherein the Nth order stationary wavelet transform comprises a 7^{th} order transform using a Haar wavelet.

Example 7A. The system of any of examples 1A-6A, wherein the wavelet transform is an Nth order stationary wavelet transform, and wherein the processing circuitry is configured to process the high frequency noise and as part of processing the high frequency noise, the processing circuitry is configured to: pre-process device-determined beat markers or re-compute beat markers; remove an ECG signal mean or median from the sensed ECG signal; and except within a predetermined number of samples around each beat marker location, reduce an amplitude of all other first and second level detail samples.

Example 8A. The system of example 7A, wherein the Nth order stationary wavelet transform comprises a 7^{th} order transform using a Haar wavelet.

Example 9A. The system of any of examples 1A-8A, wherein the processing circuitry is further configured to receive, from a clinician via a user interface, an indication of a selected level of processing of the at least one of the low frequency noise or the high frequency noise in the sensed ECG signal, wherein the processing the at least one of low frequency noise or high frequency noise in the sensed ECG signal is based, at least in part, on the indication.

Example 10A. The system of any of examples 1A-9A, wherein the processing circuitry is further configured to receive an indication of a processing the at least one of low frequency noise or high frequency noise in the sensed ECG signal is based, at least in part, on the indication.

Example 11A. The system of example 10A, wherein the processing circuitry is further configured to at least one of: determine whether the indication is of a device-detected atrial fibrillation (AF) episode, wherein if the indication is of the device-detected AF episode, the processing circuitry is configured to process the high frequency noise and process the low frequency noise after processing the high frequency noise; or determine whether the indication is of a device-detected pause, bradycardia, tachycardia, or atrial tachycardia, wherein if the indication is of the device-detected pause, bradycardia, tachycardia, or atrial tachycardia, the processing circuitry is configured to process the low frequency noise and process the high frequency noise after processing the low frequency noise.

Example 12A. The system of any of examples 1A-11A, wherein the processing circuitry is further configured to: present to a clinician a set of representative processed ECG signals, the set of representative processed ECG signals comprising a plurality of processed ECG signals being processed differently; and receive clinician visualization ratings for one or more of the plurality of processed ECG signals, wherein the processing of the at least one of low frequency noise or high frequency noise in the sensed ECG signal is based, at least in part, on the clinician visualization ratings.

Example 13A. The system of any of examples 1A-12A, wherein the processing circuitry is further configured to: present to a clinician a set of representative processed ECG signals, the set of representative processed ECG signals comprising a plurality of processed ECG signals of a particular patient being processed differently; and receive patient visualization ratings from a clinician for one or more of the plurality of processed ECG signals, wherein the processing of the at least one of low frequency noise or high frequency noise in the sensed ECG signal is based, at least in part, on the patient visualization ratings.

Example 14A. The system of any of examples 1A-13A, wherein as part of generating the processed ECG signal, the processing circuitry is configured to at least one of normalize at least one R-wave amplitude or amplify at least one P-wave amplitude.

Example 15A. The system of any of examples 1A-14A, wherein the sensed ECG signal is sensed by an insertable cardiac monitor via a plurality of subcutaneous electrodes.

Example 16A. A method of processing a sensed electrocardiogram (ECG) signal to change a visualization of the sensed ECG signal, the method comprising: receiving the sensed ECG signal; processing at least one of low frequency noise or high frequency noise in the sensed ECG signal to generate a processed ECG signal in which the at least one of the low frequency noise or the high frequency noise is mitigated, wherein the ECG signal includes a plurality of waves, and the processing of the at least one of the low frequency noise or the high frequency noise preserves a morphology of the waves, and wherein as part of processing the at least one of low frequency noise or high frequency noise in the sensed ECG signal, the processing circuitry is configured to determine one or more wavelet transforms to the ECG signal; and outputting the processed ECG signal with the at least one of the low frequency noise or the high frequency noise mitigated and the morphology of the waves preserved for visualization by a clinician for diagnosis by the clinician of a cardiac episode.

Example 17A. The method of example 16A, wherein processing the at least one of the low frequency noise or the high frequency noise comprises: determining a wavelet transform of the ECG signal; processing the wavelet transform; and determining an inverse wavelet transform of the processed wavelet transform to generate the processed ECG signal.

Example 18A. The method of example 16A or example 17A, further comprising determining that one or more visualization criterion associated with the sensed ECG signal are met, wherein the processing the at least one of the low frequency noise or the high frequency noise in the sensed ECG signal to generate the processed ECG signal is based on the one or more visualization criterion associated with the sensed ECG signal being met.

Example 19. The method of example 18A, wherein the one or more visualization criterion comprise at least one of an amplitude of the low frequency noise meeting a threshold or an amplitude of the high frequency noise meeting a threshold.

Example 20A. The method of any of examples 16A-19A, wherein the wavelet transform is an Nth order stationary wavelet transform, and wherein the method comprises processing the low frequency noise, and wherein processing the low frequency noise comprises: removing an ECG signal mean or median from the sensed ECG signal; and reducing an amplitude of Nth level approximation samples.

Example 21A. The method of example 20A, wherein the Nth order stationary wavelet transform comprises a 7^{th} order transform using a Haar wavelet.

Example 22A. The method of any of examples 16A-22A, wherein the wavelet transform is an Nth order stationary wavelet transform, wherein the method comprises processing the high frequency noise, and wherein processing the high frequency noise comprises: pre-processing device-determined beat markers or re-computing beat markers; removing an ECG signal mean or median from the sensed ECG signal; and except within a predetermined number of samples around each beat marker location, reducing an amplitude of all other first and second level detail samples.

Example 23A. The method of example 22A, wherein the Nth order stationary wavelet transform comprises a 7^{th} order transform using a Haar wavelet.

Example 24A. The method of any of examples 16A-23A, further comprising receiving, from a clinician via a user interface, an indication of a selected level of processing of the at least one of the low frequency noise or the high frequency noise in the sensed ECG signal, wherein the processing the at least one of low frequency noise or high frequency noise in the sensed ECG signal is based, at least in part, on the indication.

Example 25A. The method of any of examples 16A-24A, further comprising receiving an indication of a type of the device-detected cardiac episode, wherein the processing the at least one of low frequency noise or high frequency noise in the sensed ECG signal is based, at least in part, on the indication.

Example 26A. The method of example 25A, further comprising at least one of: determining whether the indication is of a device-detected atrial fibrillation (AF) episode, wherein if the indication is of the device-detected AF episode, processing the high frequency noise and processing the low frequency noise after processing the high frequency noise; or determining whether the indication is of a device-detected pause, bradycardia, tachycardia, or atrial tachycardia, wherein if the indication is of the device-detected pause, bradycardia, tachycardia, or atrial tachycardia, processing the low frequency noise and processing the high frequency noise after processing the low frequency noise.

Example 27A. The method of any of examples 16A-26A, further comprising: presenting to a clinician a set of representative processed ECG signals, the set of representative processed ECG signals comprising a plurality of processed ECG signals being processed differently; and receiving clinician visualization ratings for one or more of the plurality of processed ECG signals, wherein the processing of the at least one of low frequency noise or high frequency noise in the sensed ECG signal is based, at least in part, on the clinician visualization ratings.

Example 28A. The method of any of examples 16A-27A, further comprising: presenting to a clinician a set of representative processed ECG signals, the set of representative processed ECG signals comprising a plurality of processed ECG signals of a particular patient being processed differently; and receiving patient visualization ratings from a clinician for one or more of the plurality of processed ECG signals, wherein the processing of the at least one of low frequency noise or high frequency noise in the sensed ECG signal is based, at least in part, on the patient visualization ratings.

Example 29A. The method of any of examples 16A-28A, wherein generating the processed ECG signal further comprises at least one of normalizing at least one R-wave amplitude or amplifying at least one P-wave amplitude.

Example 30A. The method of any of examples 16A-29A, wherein the sensed ECG signal is sensed by an insertable cardiac monitor via a plurality of subcutaneous electrodes.

Example 31A. A non-transitory computer-readable storage medium storing instructions, which when executed, cause processing circuitry to: receive a sensed ECG signal; process at least one of low frequency noise or high frequency noise in the sensed ECG signal to generate a processed ECG signal in which the at least one of the low frequency noise or the high frequency noise is mitigated, wherein the ECG signal includes a plurality of waves, and the processing of the at least one of the low frequency noise or the high frequency noise preserves a morphology of the waves, and wherein as part of processing the at least one of low frequency noise or high frequency noise in the sensed ECG signal, the processing circuitry is configured to determine one or more wavelet transforms to the ECG signal; and output the processed ECG signal for visualization by a clinician for diagnosis of a cardiac episode.

Example 1B. A system for processing a sensed electrocardiogram (ECG) signal to change a visualization of the sensed ECG signal, the system comprising: a memory configured to store the sensed ECG signal; and processing circuitry communicatively coupled to the memory, the processing circuitry being configured to: receive the sensed ECG signal; at least one of normalize at least one R-wave of the sensed ECG signal or amplify at least one P-wave of the sensed ECG signal to generate a processed ECG signal, wherein to normalize the at least one R-wave, the processing circuitry is configured to apply a first amplitude gain to the sensed ECG signal, the first amplitude gain based on an R-wave amplitude deviation, and wherein to amplify the at least one P-wave, the processing circuitry is configured to apply a second amplitude gain to the sensed ECG signal, the second amplitude gain based on a sensed ECG signal deviation; and output the processed ECG signal for visualization by a clinician for diagnosis of a cardiac episode.

Example 2B. The system of example 1B, wherein the processing circuitry is further configured to determine that one or more visualization criterion associated with the sensed ECG signal are met, wherein the at least one of normalizing the at least one R-wave of the sensed ECG signal or amplifying the at least one P-wave of the sensed ECG signal to generate a processed ECG signal is based on the one or more visualization criterion associated with the sensed ECG signal being met.

Example 3B. The system of example 2B, wherein the one or more visualization criterion comprise at least one of an R-wave amplitude deviation level meeting a threshold or an amplitude of a P-wave meeting a threshold.

Example 4B. The system of any of examples 1B-3B, wherein the processing circuitry is configured to normalize at least one R-wave and as part of normalizing the at least one R-wave, the processing circuitry is configured to: remove an ECG signal mean or median from the sensed ECG signal; determine a first signal envelope of the sensed ECG signal and determine an R-wave amplitude deviation, the R-wave amplitude deviation comprising a difference between a largest and a smallest signal envelope deviations; determine that the R-wave amplitude deviation meets a threshold; determine a plurality of signal envelopes using moving windows; determine a respective R-wave amplitude deviation for each moving window; determine a smallest R-wave amplitude deviation of the respective R-wave amplitude deviations and set a maximum R-wave amplitude deviation of the processed ECG signal to be equal to a multiple of the smallest R-wave amplitude deviation; determine that the respective R-wave amplitude deviation is less than the maximum R-wave amplitude deviation; set an R-wave amplitude deviation of the processed ECG signal to equal the respective R-wave amplitude deviation; and apply a gain to the processed ECG signal.

Example 5B. The system of any of examples 1B-3B, wherein the processing circuitry is configured to normalize at least one R-wave and as part of normalizing the at least one R-wave, the processing circuitry is configured to: remove an ECG signal mean or median from the sensed ECG signal; determine a first signal envelope of the sensed ECG signal and determine an R-wave amplitude deviation, the determined ECG signal R-wave amplitude deviation comprising a difference between a largest and a smallest signal envelope deviations; determine that the R-wave amplitude deviation level meets a threshold; determine a plurality of signal envelopes using moving windows; determine a respective R-wave amplitude deviation for each moving window; determine a smallest R-wave amplitude deviation of the respective R-wave amplitude deviations and set a maximum R-wave amplitude deviation of the processed ECG signal to be equal to a multiple of the smallest R-wave amplitude deviation; determine that a respective R-wave amplitude deviation is not less than the maximum R-wave amplitude deviation; cap an R-wave amplitude deviation of the processed ECG signal to the maximum R-wave amplitude deviation; and apply a gain to the processed ECG signal.

Example 6B. The system of example 4B or example 5B, wherein as part of determining the first signal envelope of the sensed ECG signal, the processing circuitry is configured to apply a moving window to the sensed ECG signal, the moving window having an 8 second length and a 1 second movement.

Example 7B. The system of any of examples 1B-6B, wherein the processing circuitry is configured to amplify at least one P-wave of the sensed ECG signal and as part of amplifying the at least one P-wave, the processing circuitry is configured to: remove an ECG signal mean or median from the sensed ECG signal; rescale the sensed ECG signal to a predetermined range; determine a second signal envelope; determine a signal deviation amplitude; determine a rescaled signal deviation amplitude; determine a gain based on the rescaled signal deviation amplitude; and multiply the sensed ECG signal by the determined gain.

Example 8B. The system of example 7B, wherein as part of determining the second signal envelope of the sensed ECG signal, the processing circuitry is configured to apply a moving window to the sensed ECG signal, the moving window having a 62.5 ms length and a 62.5 ms movement.

Example 9B. The system of example 7B or example 8B, wherein as part of determining the rescaled signal deviation amplitude, the processing circuitry is configured to apply: Rescaled signal deviation amplitude=A+ (K−A)/(C+Q e^{-B*(original signal deviation)})^{1/v} where A=0, K=1, C=1, B=10, Q=0.5, and v=0.1.

Example 10B. The system of any of examples 7B-9B, wherein as part of determining the gain, the processing circuitry is configured to apply the formula:

Gain=0.8/(0.8+rescaled signal deviation amplitude).

Example 11B. The system of any of examples 1B-10B, wherein the processing circuitry is further configured to receive an indication of a type of the device-detected cardiac episode, wherein the processing the at least one of low frequency noise or high frequency noise in the sensed ECG signal is based, at least in part, on the indication.

Example 12B. The system of example 11B, wherein the processing circuitry is further configured: determine that the indication is of a device-detected atrial fibrillation (AF) episode, pause, bradycardia, tachycardia, or atrial tachycardia; and based on the indication being of a device-detected atrial fibrillation (AF) episode, pause, bradycardia, tachycardia, or atrial tachycardia, normalize the at least one R-wave.

Example 13B. The system of any of examples 1B-12B, wherein the processing circuitry is further configured to: present to a clinician a set of representative processed ECG signals, the set of representative processed ECG signals comprising a plurality of processed ECG signals being processed differently; and receive clinician visualization ratings for one or more of the plurality of processed ECG signals, wherein the at least one of normalizing of the at least one R-wave or amplifying the at least one P-wave in the sensed ECG signal is based, at least in part, on the clinician visualization ratings.

Example 14B. The system of any of examples 1B-13B, wherein the processing circuitry is further configured to: present to a clinician a set of representative processed ECG signals, the set of representative processed ECG signals comprising a plurality of processed ECG signals of a particular patient being processed differently; and receive patient visualization ratings from a clinician for one or more of the plurality of processed ECG signals, wherein the at least one of normalizing of the at least one R-wave or amplifying the at least one P-wave in the sensed ECG signal in the sensed ECG signal is based, at least in part, on the patient visualization ratings.

Example 15B. The system of any of examples 1B-14B, wherein as part of generating the processed ECG signal, the processing circuitry is configured to process at least one of low frequency noise or high frequency noise in the sensed ECG signal.

Example 16B. The system of any of examples 1B-15B, wherein the sensed ECG signal is sensed by an insertable cardiac monitor via a plurality of subcutaneous electrodes.

Example 17B. A method of processing a sensed electrocardiogram (ECG) signal to change a visualization of the sensed ECG signal, the method comprising: receiving the sensed ECG signal; at least one of normalizing at least one R-wave of the sensed ECG signal or amplifying at least one P-wave of the sensed ECG signal to generate a processed ECG signal, wherein normalizing the at least one R-wave comprises applying a first amplitude gain to the sensed ECG signal, the first amplitude gain based on an R-wave amplitude deviation, and wherein amplifying the at least one P-wave comprises applying a second amplitude gain to the sensed ECG signal, the second amplitude gain based on a sensed ECG signal deviation; and outputting the processed ECG signal for visualization by a clinician for diagnosis of a cardiac episode.

Example 18B. The method of example 17B, further comprising determining that one or more visualization criterion associated with the sensed ECG signal are met, wherein the at least one of the normalizing the at least one R-wave of the sensed ECG signal or the amplifying the at least one P-wave of the sensed ECG signal to generate a processed ECG signal is based on the one or more visualization criterion associated with the sensed ECG signal being met.

Example 19B. The method of example 18B, wherein the one or more visualization criterion comprise at least one of an R-wave amplitude deviation level meeting a threshold or an amplitude of a P-wave meeting a threshold.

Example 20B. The method of any of examples 17B-19B, wherein normalizing the at least one R-wave comprises: removing an ECG signal mean or median from the sensed ECG signal; determining a first signal envelope of the sensed ECG signal and determine an R-wave amplitude deviation, the R-wave amplitude deviation comprising a difference between a largest and a smallest signal envelope deviations; determining that the R-wave amplitude deviation meets a threshold; determining a plurality of signal envelopes using moving windows; determining a respective R-wave amplitude deviation for each moving window; determining a smallest R-wave amplitude deviation of the respective R-wave amplitude deviations and set a maximum R-wave amplitude deviation of the processed ECG signal to be equal to a multiple of the smallest R-wave amplitude deviation; determining that a respective R-wave amplitude deviation is less than the maximum R-wave amplitude deviation; setting an R-wave amplitude deviation of the processed ECG signal to equal the respective R-wave amplitude deviation; and applying a gain to the processed ECG signal.

Example 21B. The method of any of examples 17B-19B, wherein normalizing the at least one R-wave comprises: removing an ECG signal mean or median from the sensed ECG signal; determining a first signal envelope of the sensed ECG signal and determine an R-wave amplitude deviation, the determined ECG signal R-wave amplitude deviation comprising a difference between a largest and a smallest signal envelope deviations; Determining that the R-wave amplitude deviation level meets a threshold; determining a plurality of signal envelopes using moving windows; determining a respective R-wave amplitude deviation for each moving window; determining a smallest R-wave amplitude deviation of the respective R-wave amplitude deviations and set a maximum R-wave amplitude deviation of the processed ECG signal to be equal to a multiple of the smallest R-wave amplitude deviation; determining that a respective R-wave amplitude deviation is not less than the maximum R-wave amplitude deviation; capping an R-wave amplitude deviation of the processed ECG signal to the maximum R-wave amplitude deviation; and applying a gain to the processed ECG signal.

Example 22B. The method of example 20B or example 21B, wherein determining the first signal envelope of the sensed ECG signal comprises applying a moving window to the sensed ECG signal, the moving window having an 8 second length and a 1 second movement.

Example 23B. The method of any of examples 17B-22B, wherein amplifying the at least one P-wave of the sensed ECG signal comprises: removing an ECG signal mean or median from the sensed ECG signal; rescaling the sensed ECG signal to a predetermined range; determining a second signal envelope; determining a signal deviation amplitude; determining a rescaled signal deviation amplitude; determining a gain based on the rescaled signal deviation amplitude; and multiplying the sensed ECG signal by the determined gain.

Example 24B. The method of example 23B, wherein determining the second signal envelope of the sensed ECG signal comprises applying a moving window to the sensed ECG signal, the moving window having a 62.5 ms length and a 62.5 ms movement.

Example 25B. The method of example 23B or example 24B, wherein as part of determining the rescaled signal deviation amplitude, the processing circuitry is configured to apply: Rescaled signal deviation amplitude=A+ (K−A)/(C+Q e^{-B*(original signal deviation)})^{1/v} where A=0, K=1, C=1, B=10, Q=0.5, and v=0.1.

Example 26B. The method of any of examples 23B-25B, wherein determining the gain comprises applying: Gain=0.8/(0.8+rescaled signal deviation amplitude).

Example 27B. The method of any of examples 17B-26B, further comprising receiving an indication of a type of the device-detected cardiac episode, wherein the processing the at least one of low frequency noise or high frequency noise in the sensed ECG signal is based, at least in part, on the indication.

Example 28B. The method of example 27B, further comprising: determining that the indication is of a device-detected atrial fibrillation (AF) episode, pause, bradycardia, tachycardia, or atrial tachycardia; and based on the indication being of a device-detected atrial fibrillation (AF) episode, pause, bradycardia, tachycardia, or atrial tachycardia, normalizing the at least one R-wave.

Example 29B. The method of any of examples 17B-28B, further comprising: presenting to a clinician a set of representative processed ECG signals, the set of representative processed ECG signals comprising a plurality of processed ECG signals being processed differently; and receiving clinician visualization ratings for one or more of the plurality of processed ECG signals, wherein the at least one of normalizing of the at least one R-wave or amplifying the at least one P-wave in the sensed ECG signal is based, at least in part, on the clinician visualization ratings.

Example 30B. The method of any of examples 17B-29B, further comprising: presenting to a clinician a set of representative processed ECG signals, the set of representative processed ECG signals comprising a plurality of processed ECG signals of a particular patient being processed differently; and receiving patient visualization ratings from a clinician for one or more of the plurality of processed ECG signals, wherein the at least one of normalizing of the at least one R-wave or amplifying the at least one P-wave in the sensed ECG signal in the sensed ECG signal is based, at least in part, on the patient visualization ratings.

Example 31B. The method of any of examples 17B-30B, wherein generating the processed ECG signal further comprises processing at least one of low frequency noise or high frequency noise in the sensed ECG signal.

Example 32B. The method of any of examples 17B-31B, wherein the sensed ECG signal is sensed by an insertable cardiac monitor via a plurality of subcutaneous electrodes.

Example 33B. A non-transitory computer-readable storage medium storing instructions, which when executed, cause processing circuitry to: receive a sensed ECG signal; at least one of normalize at least one R-wave of the sensed ECG signal or amplify at least one P-wave of the sensed ECG signal to generate a processed ECG signal, wherein to normalize the at least one R-wave, instructions cause the processing circuitry to apply a first amplitude gain to the sensed ECG signal, the first amplitude gain based on an R-wave amplitude deviation, and wherein to amplify the at least one P-wave, the instructions cause the processing circuitry to apply a second amplitude gain to the sensed ECG signal, the second amplitude gain based on a sensed ECG signal deviation; and output the processed ECG signal for visualization by a clinician for diagnosis of a cardiac episode.

Various examples have been described. These and other examples are within the scope of the following claims.

1. A system for processing a sensed electrocardiogram (ECG) signal to change a visualization of the sensed ECG signal, the system comprising:
a memory configured to store the sensed ECG signal; and
processing circuitry communicatively coupled to the memory, the processing circuitry being configured to:
receive the sensed ECG signal;
process at least one of low frequency noise or high frequency noise in the sensed ECG signal to generate a processed ECG signal in which the at least one of the low frequency noise or the high frequency noise is mitigated, wherein the ECG signal includes a plurality of waves, and the processing of the at least one of the low frequency noise or the high frequency noise preserves a morphology of the waves, and wherein as part of processing the at least one of low frequency noise or high frequency noise in the sensed ECG signal, the processing circuitry is configured to determine one or more wavelet transforms to the ECG signal; and
output the processed ECG signal with the at least one of the low frequency noise or the high frequency noise mitigated and the morphology of the waves preserved for visualization by a clinician for diagnosis of a cardiac episode.

2. The system of claim 1, wherein to process the at least one of the low frequency noise or the high frequency noise comprising determining the one more wavelet transforms, the processing circuitry is configured to:
determine a wavelet transform of the ECG signal;
process the wavelet transform; and
determine an inverse wavelet transform of the processed wavelet transform to generate the processed ECG signal.

3. The system of claim 1, wherein the processing circuitry is further configured to determine that one or more visualization criterion associated with the sensed ECG signal are met, wherein processing the at least one of the low frequency noise or the high frequency noise in the sensed ECG signal to generate the processed ECG signal is based on the one or more visualization criterion associated with the sensed ECG signal being met.

4. The system of claim 3, wherein the one or more visualization criterion comprises at least one of an amplitude of the low frequency noise meeting a threshold or an amplitude of the high frequency noise meeting a threshold.

5. The system of claim 1, wherein the wavelet transform is an N^{th} order stationary wavelet transform, and wherein the processing circuitry is configured to process the low frequency noise, and as part of processing the low frequency noise, the processing circuitry is configured to:
remove an ECG signal mean or median from the sensed ECG signal; and
reduce an amplitude of N^{th} level approximation samples.

6. The system of claim 5, wherein the N^{th} order stationary wavelet transform comprises a 7^{th} order transform using a Haar wavelet.

7. The system of claim 1, wherein the wavelet transform is an N^{th} order stationary wavelet transform, and wherein the processing circuitry is configured to process the high frequency noise and as part of processing the high frequency noise, the processing circuitry is configured to:
pre-process device-determined beat markers or re-compute beat markers;
remove an ECG signal mean or median from the sensed ECG signal; and
except within a predetermined number of samples around each beat marker location, reduce an amplitude of all other first and second level detail samples.

8. The system of claim 7, wherein the N^{th} order stationary wavelet transform comprises a 7^{th} order transform using a Haar wavelet.

9. The system of claim 1, wherein the processing circuitry is further configured to receive, from a clinician via a user interface, an indication of a selected level of processing of the at least one of the low frequency noise or the high frequency noise in the sensed ECG signal, wherein the processing the at least one of low frequency noise or high frequency noise in the sensed ECG signal is based, at least in part, on the indication.

10. The system of claim 1, wherein the processing circuitry is further configured to receive an indication, and wherein the processing the at least one of low frequency noise or high frequency noise in the sensed ECG signal is based, at least in part, on the indication.

11. The system of claim 10, wherein the processing circuitry is further configured to at least one of:
determine whether the indication is of a device-detected atrial fibrillation (AF) episode, wherein if the indication is of the device-detected AF episode, the processing circuitry is configured to process the high frequency noise and process the low frequency noise after processing the high frequency noise; or
determine whether the indication is of a device-detected pause, bradycardia, tachycardia, or atrial tachycardia, wherein if the indication is of the device-detected pause, bradycardia, tachycardia, or atrial tachycardia, the processing circuitry is configured to process the low frequency noise and process the high frequency noise after processing the low frequency noise.

12. The system of claim 1, wherein the processing circuitry is further configured to:
present to a clinician a set of representative processed ECG signals, the set of representative processed ECG signals comprising a plurality of processed ECG signals being processed differently; and
receive clinician visualization ratings for one or more of the plurality of processed ECG signals, wherein the processing of the at least one of low frequency noise or high frequency noise in the sensed ECG signal is based, at least in part, on the clinician visualization ratings.

13. The system of claim 1, wherein the processing circuitry is further configured to:
present to a clinician a set of representative processed ECG signals, the set of representative processed ECG signals comprising a plurality of processed ECG signals of a particular patient being processed differently; and
receive patient visualization ratings from a clinician for one or more of the plurality of processed ECG signals, wherein the processing of the at least one of low frequency noise or high frequency noise in the sensed ECG signal is based, at least in part, on the patient visualization ratings.

14. A method of processing a sensed electrocardiogram (ECG) signal to change a visualization of the sensed ECG signal, the method comprising:
receiving the sensed ECG signal;
processing at least one of low frequency noise or high frequency noise in the sensed ECG signal to generate a processed ECG signal in which the at least one of the low frequency noise or the high frequency noise is mitigated, wherein the ECG signal includes a plurality of waves, and the processing of the at least one of the low frequency noise or the high frequency noise preserves a morphology of the waves, and wherein processing the at least one of low frequency noise or high frequency noise in the sensed ECG signal comprises determining one or more wavelet transforms to the ECG signal; and
outputting the processed ECG signal with the at least one of the low frequency noise or the high frequency noise mitigated and the morphology of the waves preserved for visualization by a clinician for diagnosis by the clinician of a cardiac episode.

15. A non-transitory computer-readable storage medium storing instructions, which when executed, cause processing circuitry to:
receive a sensed electrocardiogram (ECG) signal;
process at least one of low frequency noise or high frequency noise in the sensed ECG signal to generate a processed ECG signal in which the at least one of the low frequency noise or the high frequency noise is mitigated, wherein the ECG signal includes a plurality of waves, and the processing of the at least one of the low frequency noise or the high frequency noise preserves a morphology of the waves, and wherein as part of processing the at least one of low frequency noise or high frequency noise in the sensed ECG signal, the processing circuitry is configured to determine one or more wavelet transforms to the ECG signal; and
output the processed ECG signal for visualization by a clinician for diagnosis of a cardiac episode.

16. The system of claim 1, wherein as part of generating the processed ECG signal, the processing circuitry is configured to at least one of normalize at least one R-wave amplitude or amplify at least one P-wave amplitude.

17. The system of claim 1, wherein the sensed ECG signal is sensed by an insertable cardiac monitor via a plurality of subcutaneous electrodes.

18. The method of claim 14, wherein processing the at least one of the low frequency noise or the high frequency noise comprises:
determining a wavelet transform of the ECG signal;
processing the wavelet transform; and
determining an inverse wavelet transform of the processed wavelet transform to generate the processed ECG signal.

19. The method of claim 14, further comprising determining that one or more visualization criterion associated with the sensed ECG signal are met, wherein the processing the at least one of the low frequency noise or the high frequency noise in the sensed ECG signal to generate the processed ECG signal is based on the one or more visualization criterion associated with the sensed ECG signal being met.

20. The method of claim 19, wherein the one or more visualization criterion comprise at least one of an amplitude of the low frequency noise meeting a threshold or an amplitude of the high frequency noise meeting a threshold.
